(19) United States
(12) Patent Application Publication
(10) Pub. No.: US 2023/0355675 A1
(43) Pub. Date: Nov. 9, 2023

(54) COMPOSITIONS AND METHODS FOR PRODUCING AND USING ILCS TO TREAT HEALTH CONDITIONS

(71) Applicant: THE REGENTS OF THE UNIVERSITY OF COLORADO, A BODY CORPORATE, Denver, CO (US)

(72) Inventors: Michael Verneris, Greenwood Village, CO (US); Dejene Tufa, Aurora, CO (US)

(21) Appl. No.: 18/127,284

(22) Filed: Mar. 28, 2023

Related U.S. Application Data
(63) Continuation of application No. PCT/US2021/052449, filed on Sep. 28, 2021.
(60) Provisional application No. 63/084,440, filed on Sep. 28, 2020.

(51) Int. Cl. A61K 35/17; C12N 5/078

(52) U.S. Cl. CPC A61K 35/17; C12N 5/0634; C12N 2502/1394

(57) ABSTRACT

Embodiments of the instant disclosure relate to novel compositions, methods and systems for generating ILC cells. In certain embodiments, the present disclosure provides for a composition including a hematopoietic progenitor cell expressing CD48 and at least one of a CD48 ligand, a CD48 agonist or a CD48 antagonist in order to induce production of ILC2 or ILC3 (for example, NCR⁺ ILC3 and LTi-ILC3) cell populations. In other certain embodiments, the present disclosure provides methods of treating one or more health condition or immune-mediated condition in a subject by administering an effective amount of a composition of ILC2 or ILC3 cells generated using methods disclosed herein.

### PRIORITY

This application is a Continuation Application of International Application No. PCT/2021/052449, filed on Sep. 28, 2021, which claims the benefit of priority pursuant to 35 U.S.C. § 119(e) to U.S. Provisional Application No. 63/084,440 filed on Sep. 28, 2020. These applications are incorporated herein by reference in their entirety for all purposes.

### GOVERNMENT FUNDING

This invention was made with government support under Grant No. RO1 A100879-05 awarded by the National Institutes of Health (NIH). The government has certain rights in this invention.

### FIELD

Embodiments of the instant disclosure generally relate to novel compositions, methods and systems for generating innate lymphoid cells (ILCS) such as ILC2s and ILC3s and uses thereof.

### BACKGROUND OF THE INVENTION

Innate lymphoid cells (ILCs) are a family of immune cells that have features of innate and adaptive immunity. Therefore, these cells promptly respond to infections and other signals similar to innate immune cells, but at the same time secrete a cytokine pattern profile similar to T cells despite having no T cell receptor. ILCs are tissue resident lymphocytes that play diverse roles in lymphoid tissue formation, immunity, inflammation and tissue remodeling in addition to overall contributions to both health and disease.

ILCs are subdivided by the transcription factors they express and the cytokines they produce. ILC1s are inflammatory cells that are known to play a role in tissue defenses. ILC2s are involved in intestinal health (e.g. expressed during a parasitic infection), pulmonary health and disease, renal health, cardiovascular health, and other roles. ILC3s are mainly intestinal lymphocytes that play a role in intestinal homeostasis but also play a role in lymph node generation and repair. In addition, ILC1s, ILC2s and ILC3s have several subtypes for each class.

ILCs arise from common lymphoid progenitors (CLPs) in the bone marrow however the extrinsic signals and transcriptional changes required for the progressive lineage restriction of CLPs to common ILC progenitors, and subsequently to immature precursors of the individual ILC lineages are not fully understood.

Recent studies have explored harnessing a subject’s own immune cells, using partially related or unrelated immune cells to mediate antitumor and antiviral responses by employing adoptive cell transfer. ILCs could be a candidate for adoptive cell transfer regimens; however, because these cells are tissue resident (and minimally circulate); obtaining sufficient numbers of ILCs has been problematic. Therefore, there is a need to produce ILCs in suitable numbers in vitro for therapeutic adoptive cell transfer and/or other therapies.

### SUMMARY

Embodiments of the instant disclosure relate to novel compositions, methods and systems for generating ILC2 cells. In some embodiments, compositions disclosed herein can include hematopoietic progenitor cells expressing CD48 and its receptor CD244, and a cell or cell population having at least one of a CD48 ligand or a CD244 agonist are provided for inducing production of ILC2 cells. In certain embodiments, compositions disclosed herein can further include at least one cytokine or growth factor. In some embodiments, the at least one cytokine or growth factor included in the disclosed composition can stimulate hematopoietic progenitor cell differentiation. In accordance with these embodiments, a cytokine or growth factor can include, but is not limited to, stem cell factor (SCF), interleukin 3 (IL-3), interleukin 7 (IL-7), interleukin 15 (IL-15), interleukin 23 (IL-23), IL-2, IL-25, IL-33 FMS-like tyrosine kinase 3 ligand (FLT3L), or a combination thereof. In certain embodiments, methods of producing ILC2 and/or ILC3 cells (e.g. LTi-ILC3) are disclosed and other methods are described for inducing preferred differentiation of ILC2 and/or ILC3 (e.g. LTi-ILC3) cells for production and use in the treatment of mammalian health conditions.

In some embodiments, a hematopoietic progenitor cell expressing CD48 disclosed herein can further express at least one other marker including, but not limited to, CD34, α4β7, CD52, AMICA1, CCR7, CD44, CD53, CD63, CD99, CD117, CD127, HLA-A, IL2RG, KRT1, NOTCH½, IL-2/ IL-2R, IL-25/IL17BR and IL-33/IL1RL1/ST2 or a combination thereof. In some embodiments, a hematopoietic progenitor cell expressing CD48 disclosed herein can further express at least one other marker including, but not limited to, CD34 and α4β7 but be CD52 negative (CD52-) and give rise to LTi-ILC3 cells. In accordance with these embodiments, the LTi-ILC3s produced by methods disclosed herein can be used in the treatment of health conditions.

In certain embodiments, compositions disclosed herein can further include stroma having certain capabilities of use for generating ILC2 cells. In other embodiments, stroma of use herein can express or over-express CD48. In yet other embodiments, the stroma herein can be irradiated stroma. In accordance with these embodiments, stroma can be derived from bone marrow, cell lines or fibroblasts or other suitable source. In yet other embodiments, stroma of used herein can be combined with HPCs in order to generate ILC2s and ILC3s of use in therapeutic methods known in the art.

In certain embodiments, the at least one CD48 ligand or CD244 agonist of the compositions disclosed herein can be a small molecule, a polypeptide or fragment thereof, a polynucleotide, genetically modified or synthesized molecule or an antibody or a fragment thereof or a combination thereof. In accordance with these embodiments, a CD48 ligand or CD244 agonist includes, but is not limited to, agonist CD48 antibody, soluble CD244 or CD244 mimetic, CD244 fixed to a solid material such as a matrix or beads, chimeric antigen receptors binding CD244 (CD244 CAR), an agent that modulates or interferes with CD244/2B4 interactions or a mimetic thereof, cells capable of expressing agonist CD48 antibody, or any CD48 ligand or CD244 agonist known in the art.

In some embodiments, compositions disclosed herein can include a hematopoietic progenitor cell that expresses CD244 receptors where the at least one ligand (e.g. CD48 ligand) or CD244 agonist modulates activation of the CD244 receptor and/or modulates downstream effects in order to induce differentiation in HPCs of use herein. In some embodiments, activation of CD244 can lead to downstream signaling and production of selected cells disclosed herein.

In some embodiments, methods for producing ILC2 cells (or ILC2s) are disclosed. In certain embodiments, methods disclosed herein include providing to a hematopoietic progenitor cell expressing CD48 and CD244, a composition including, but not limited to, at least one of a CD48/CD244 ligand or a CD244 agonist. In other embodiments, methods disclosed herein, can further include seeding the hematopoietic progenitor cell expressing CD48 and CD244 onto a stromal cell line as disclosed herein in order to at least accelerate ILC2 production and produce at least 5%, or 10% or 15% or 20% or more ILC2 cells than produced under naturally occurring conditions such as without use of a stomal cell line (e.g. expressing CD48). In other embodiments, ILC2 cells produced by methods disclosed herein can be harvested and immediately used, refrigerated or frozen for later use.

In some embodiments, methods disclosed herein yield ILC2 cells wherein the ILC2 cells can express CD11a, CD117, or a combination thereof. In certain embodiments, methods disclosed herein yield an enriched population of cells containing ILC2 cells, wherein at least 20%, at least 30%, at least 40%, at least 50%, at least 60%, at least 70%, at least 80% or more of the cells include viable ILC2 cells. In other embodiments, a hematopoietic progenitor cell expressing CD48 and CD244 disclosed herein can further express at least one other marker including, but not limited to, CD34 and α4β7 but be CD52 negative (CD52-) and give rise to LTi-ILC3 cells. In certain embodiments, methods disclosed herein yield an enriched population of cells containing LTi-ILC3 cells, wherein at least 20%, at least 30%, at least 40%, at least 50%, at least 60%, at least 70%, at least 80% or more of the cells contain viable LTi-ILC3 cells. In other embodiments, these enriched populations of cells can be further purified to produce a near homogenous to homogenous populations of ILC2, NCR+ ILC3 or LTi-ILC3 cells.

In other embodiments, ILC2, NCR+ ILC3 and/or LTi-ILC3 generated by compositions and methods disclosed herein can be used for treating one or more health condition in a subject in need of such a treatment. In certain embodiments, methods for treating one or more health conditions can include, but are not limited to, immune-mediated conditions or an infection. In accordance with these embodiments, an immune-mediated condition can include, but is not limited to, graft versus host disease (GvHD), inflammatory bowel diseases (IBD), Crohn’s disease, Type-1 diabetes, psoriasis, asthma, allergic responses, rheumatoid arthritis, ankylosing spondylitis, psoriatic arthritis, Behcet’s disease, cardiovascular disease or associated condition, renal disease, injury or other renal condition, or other immune-mediated disorder or a combination thereof. In other embodiments, an infection can be treated with ILC2 cells generated by compositions and methods disclosed herein. Infections can include but is not limited to an infection by a microorganism such as a virus, bacterial, parasite or fungus. In other embodiments, an immune-mediated or immuno-compromising condition to be treated disclosed herein can be cancer. In accordance with these embodiments, cancers to be treated using compositions and methods disclosed herein can be a lymphatic cancer or a lymphoma, leukemia, or other blood-related cancer or immunomodulatory cancer or a combination thereof.

### BRIEF DESCRIPTION OF THE DRAWINGS

FIGS. 1A-1J represent exemplary methods of enhancing ILC2 Development by CD244 activation. 1A illustrates representative flow cytometry data depicting the expression of CD244 and CD48 by freshly isolated CD34⁺HSCs; 1B illustrates representative flow cytometry data depicting the expression of CD244 at day -1 and -3 by CD34⁺α4β7⁺CD48⁻ and CD34⁺α4β7⁺CD48⁺; 1C illustrates a bar graph representing SAP mRNA expression; 1D and 1E illustrate bar graphs representing ILCs staining for differentiated CD34⁺α4β7⁺CD48⁺ cells in the presence of CD48 or CD244 blocking; 1D illustrates the percentage of certain NK cells, ILC2, and ILC3 cells and 1E illustrates absolute number of NK cells, ILC2, and ILC3 cells. 1F and 1G illustrate bar graphs illustrating ILCs staining for differentiated CD34⁺α4β7⁺CD48⁺ cells after CD244 signaling was activated by the addition of a cross-linking antibody during differentiation where 1F illustrates the percentage of certain NK cells, ILC2, and ILC3 cells and 1G illustrates absolute number of certain NK cells ILC2 and ILC3 cells. 1H illustrates a bar graph representing knockdown of 2B4 in transfected progenitor cells and the proportion of CD244 cell. 1I illustrates representative flow cytometry data of CD244⁺ cultures. 1J illustrates a dot plot depicting representative generation of ILC2s (control gRNA) or lack of CD244+ cells following 2B4 knock-down in some embodiments disclosed herein.

FIGS. 2A-2F represent exemplary experiments of the instant disclosure. 2A illustrates representative flow cytometry data of certain cells produced or studied herein; 2B illustrates representative flow cytometry data of other cultures under various experimental conditions disclosed herein; 2C illustrates representative flow cytometry data of ILC2s from cultures on a layer of irradiated CD48 expressing OP9 and control OP9 stromal cells. FIG. 2D illustrates representative flow cytometry data illustrating staining for CD244 from CD48⁺ cultures transfected with control gRNA (wild type) or experimental gRNA. 2E and 2F illustrate dot plots representing generation of ILC3 s (2E) and NK cells (2F) from cultures that express (control gRNA) or lack (2B4 gRNA) CD244 of some embodiments disclosed herein.

FIG. 3 represents a schematic illustration of cell differentiation and cell-surface receptor expression pathway or flow chart of progenitor HSCs to ILCs, NKs and other cells and illustrates differentiation and marker expression of ILC2 and ILC3 cells of some embodiments disclosed herein.

FIGS. 4A and 4B are representative images illustrating staining of ILCs differentiated in multi-well culture plates (A) and different cell populations expressing various cytokines (B) of some embodiments disclosed herein.

FIGS. 5A-5B illustrates an exemplary experiment of generation of ILCs from progenitor cells at different starting cell concentrations in 5A and 5B in multi-well culture plates of some embodiments disclosed herein.

FIG. 6 represents multiple dot plots illustrating staining of ILCs differentiated from CD34⁺α4β7⁺CD48⁻CD52⁻, CD34⁺α4β7⁺CD48⁻CD52⁺, CD34⁺α4β7⁺CD48⁺CD52⁻ and CD34⁺α4β7⁺CD48⁺CD52⁺ hematopoietic progenitors of some embodiments disclosed herein.

### DEFINITIONS

Terms, unless specifically defined herein, have meanings as commonly understood by a person of ordinary skill in the art relevant to certain embodiments disclosed herein or as applicable.

Unless otherwise indicated, all numbers expressing quantities of agents and/or compounds, properties such as molecular weights, reaction conditions, and as disclosed herein are contemplated as being modified in all instances by the term “about.” Accordingly, unless indicated to the contrary, the numerical parameters in the specification and claims are approximations that may vary from about 10% to about 15% plus and/or minus depending upon the desired properties sought as disclosed herein. Numerical values as represented herein inherently contain standard deviations that necessarily result from the errors found in the numerical value’s testing measurements.

As used herein, “individual”, “subject”, “host”, and “patient” can be interchangeably used herein and refer to any mammalian subject for whom diagnosis, treatment, prophylaxis or therapy is desired, for example, humans.

### DETAILED DESCRIPTION OF THE INVENTION

In the following sections, certain exemplary compositions and methods are described in order to detail certain embodiments of the invention. It will be obvious to one skilled in the art that practicing the certain embodiments does not require the employment of all or even some of the specific details outlined herein, but rather that concentrations, times and other specific details may be modified through routine experimentation. In some cases, well known methods, or components have not been included in the description.

Embodiments of the instant disclosure relate to novel compositions, methods and systems for generating ILC2, NCR+ ILC3 and/or LTi-ILC3 cells from hematopoietic stem cells (HPCs). In certain embodiments, compositions disclosed herein can include a hematopoietic progenitor cell expressing CD48, CD244 and a CD48 or CD244 modulating agent. In some embodiments, cells can be created for overexpression thereof, antibodies with agonist/antagonist properties, soluble proteins or those fixed to beads or other nanostructures, inorganic small molecule inhibitors or knock down techniques using editing such as CRISPR/Cas9 type systems known in the art. In other embodiments, compositions disclosed herein can include a hematopoietic progenitor cell expressing CD48, CD244 and a CD48 modulating agent and/or a CD244 activating agent or ligation-promoting agent thereof, including the expression by adjacent cells. In certain embodiments, compositions disclosed herein can include progenitor cells having a CD34⁺α4β7⁺CD48⁺CD52⁻ phenotype that can give rise to and can be enriched using compositions and method disclosed herein to give rise to LTi-like ILC3.

In other embodiments, compositions and/or methods disclosed herein can include progenitor cells having or expressing a CD34⁺α4β7⁺CD48⁺CD52⁻ phenotype. It is known in the art that these cells can give rise to NK, ILC1, ILC2 and NCR-ILC3 cells under certain circumstances. In certain embodiments, compositions and methods disclosed herein include ligation of CD244 (e.g. by CD48) to enrich ILC2 cell populations through induced CD244 signaling. In some embodiments, CD244 agonists and/or binding agents can be used. In other embodiments, a CD244 agonist such as an antibody (e.g. polyclonal or monoclonal) or stoma expressing or overexpressing CD48 can be used to induce enriched populations of ILC2. In certain embodiments, inducing ligation of CD244 by CD48 in progenitor cell populations disclosed herein can further enrich production of ILC2 cells.

“Hematopoietic progenitor cells (HPCs)” or “hematopoietic stem cells (HSCs)” can be used interchangeably to refer to either progenitor or stem cells present in various locations in the body (e.g. blood and bone marrow) capable of forming mature blood cells, for example, red blood cells, platelets and white blood cells. As disclosed herein, HPCs can be pluripotent cells that are then differentiated into HSCs that are capable of differentiating into several different cell types of the hematopoietic system, including, but not limited to, granulocytes, monocytes, erythrocytes, megakaryocytes, NK cells, ILCs, B-cells and T-cells. In certain embodiments, hematopoietic progenitor cells disclosed herein can be identified by expression of cell surface marker or receptor combinations known to designate these cells. In certain embodiments, HPCs can express or lack a marker or receptor including, but not limited to, CD34, α4β7, CD45, CD48, CD52, CD244, CD133, Lin (e.g. Lin⁻), Flk2, and the like for use in generating ILC2s and ILC3s as disclosed herein. In other embodiments, compositions disclosed herein can include HPCs expressing CD48 of use in compositions and methods disclosed herein to differentiate into ILCs. In other embodiments, compositions disclosed herein can include HPCs expressing CD48 and CD244 as well as include one or more of CD34, α4p7, CD52, AMICA1, CCR7, CD44, CD53, CD63, CD99, CD117, CD127, HLA-A, IL2RG, KRT1, NOTCH½, IL-2, IL-25 and IL-33 or a combination thereof. In certain embodiments, HPCs lack or have reduced expression of at least one of CD52 and Lin.

In certain embodiments, HPCs (or for example, iPSCs, induced pluripotent stem cells or reprogrammed stem cells) can be isolated, generated or harvested from a subject (e.g. allogeneic or autologous or xenogeneic from a cadaver or other species). In other embodiments, iPSCs can be generated and used to produce ILCs contemplated herein. In some embodiments, HPCs can be isolated or harvested from peripheral blood, umbilical cord blood, and/or bone marrow or other location known to harbor HPCs. In other aspects, HPCs can be isolated from peripheral blood mononuclear cells (PBMCs). In yet other embodiments, HPCs can be isolated from a leukapheresis sample. In certain embodiments, HPCs can be isolated from tumor-infiltrated lymphocytes, tissue-infiltrated lymphocytes, lymph nodes, thymus, and/or secondary lymphoid organs. Any source of HPCs whether harvested, isolated or generated is contemplated of use in compositions disclosed herein for generating targeted ILCs.

In certain embodiments, HPCs (or iPSCs) can be isolated or generated from autologous peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs. As used herein, the term “autologous” refers to peripheral blood, umbilical cord blood, bone marrow, PBMCs, leukapheresis sample, tumor-infiltrated lymphocytes, tissue-infiltrated lymphocytes, lymph nodes, thymus, tonsils and/or secondary lymphoid organs obtained from the same subject as to be treated with the compositions disclosed herein. In other embodiments, HPCs can be isolated or harvested from allogeneic peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs. As used herein, the term “allogeneic” refers to peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs obtained from a different subject of the same species as the subject to be treated with the compositions disclosed herein (e.g. a donor or cadaver). In some embodiments, HPCs can be isolated or harvested from haploidentical allogeneic peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs. In other embodiments, HPCs can be isolated or harvested from a different subject other than the subject to be treated where the HPCs can originate from any bodily location of the different subject for generating ILCs and treating the subject in need of such a treatment.

In some embodiments, compositions disclosed herein can include at least one of a CD48 and a CD244 modulating agent for use in increasing production of ILCs from progenitor cells. CD48 is a glycosyl-phosphatidyl-inositol (GPI) anchored protein expressed mainly on hematopoietic cells and exists in both a membrane-associated and a soluble form. It is one of the primary ligands that binds to CD244. As used herein, “a CD48/CD244 modulating agent” can refer to any chemical (e.g. small molecule or other chemical agent), compound, polypeptide, protein or fragment thereof, polynucleotide (DNA or RNA), or other agent that activates or inhibits CD48/CD244-mediated ligation. In accordance with these embodiments, a CD48 modulating agent can be an agent that induces CD244 ligation. In certain embodiments, a CD48 modulating agent can be a CD48 ligand capable of binding to CD244. CD48 is a low affinity ligand for CD2 and a high affinity ligand for 2B4 (CD244). CD48-2B4 interactions can modulate T cell, B cell and NK cell functions and cross-reactivity and functions. In some exemplary compositions, a CD48 modulating agent can be an agonist of CD48 and/or a CD48 stimulatory agent, capable of inducing CD48 ligation (or activation) and/or a CD244 signaling pathway or downstream activators and/or genes. In accordance with these embodiments, a ligand or agonist capable of binding or associating with CD48/CD244 can be used to direct productions of progenitor HSC cells to ILCs such as ILC2s and/or ILC3 cells (including, for example, NCR-ILC3 and LTi-ILC3).

In other embodiments, a CD48/CD244 modulating agent can be an antagonist of CD48/CD244 and/or a CD48/CD244 stimulatory agent, capable of decreasing and/or blocking CD48/CD244-related activity, for example, in a competitive or non-competitive manner (e.g. competitive binding or flooding the CD48/CD244 receptor with an antagonist to occupy the target gene binding site). In other embodiments, a CD48/CD244 modulating agent can be a polynucleotide such as, an antisense oligonucleotide to CD48/CD244, a ribozyme having catalytic activity (such as cleavage) that renders the CD48 inactive/active, an interfering RNA (RNAi) such as small interfering RNA (siRNA), or a microRNA capable of preventing or increases the expression (transcription and translation, respectively) of CD48, an antibody, an agent capable of knock-down, any peptide, antibody, small molecule or agent that interrupts CD244/2B4 interaction. In accordance with these embodiments, a polynucleotide capable of blocking CD48/CD244 and/or inducing CD48/CD244 interactions can be used to direct production of progenitor HSCs to ILCs such as ILC2s or ILC3s (including, for example, NCR-ILC3 and LTi-ILC3) as disclosed herein. Certain embodiments include, but are not limited to, tc7 antibody, antibodies that block or act as agonists, peptides or small molecules or antibodies that reduce, block or eliminate the interaction between CD48 and 2B4 or the like.

In other embodiments, antisense oligonucleotides can refer to polynucleotides having a reverse complementary sequence to a sequence of CD48 mRNA. In some embodiments, the polynucleotide can be an oligodeoxynucleotide, ribonucleotides or nucleotide analogues, or mixtures thereof. The antisense oligonucleotide can be modified in order to enhance the nuclease resistance thereof, to improve its membrane crossing capability, or both. The antisense oligonucleotide can be linear or can include one or more secondary structure(s). In other embodiments, an antisense oligonucleotide can include an enzymatic activity, such as ribozyme activity in order to act on CD48 in progenitor HSCs to generate ILCs.

By “ribozyme” as referenced herein, can include an RNA molecule which has complementarity in a target binding region to a specified gene target, for example CD48/CD244, and can also have an enzymatic activity which is active to specifically cleave target RNA. In accordance with these embodiments, this molecule is capable of catalyzing a series of reactions including the hydrolysis of phosphodiester bonds in trans (and thus can cleave other RNA molecules) under physiological conditions. It is known that enzymatic nucleic acid molecules can be targeted to an RNA transcript and achieve efficient cleavage in vitro. That is, the enzymatic RNA molecule is able of intermolecularly cleaving RNA and thereby inactivating a target RNA molecule. The complementary regions allow sufficient hybridization of the enzymatic RNA molecule to the target RNA and which ensures specific cleavage. One hundred percent complementarity is preferred, but complementarity as low as 50-75% may also be useful in this invention. The nucleic acids may be modified at the base, sugar, and/or phosphate groups.

The term “siRNAs” refers to short interfering (si) RNAs. The term “RNA interference” or “RNAi” refers to the silencing or decreasing of gene expression by siRNAs. It is the process of sequence-specific, post-transcriptional sequence-specific gene silencing in animals and plants, initiated by siRNA that is homologous in its duplex region to the sequence of the gene to be silenced, such as CD48 or CD244. The expression of the gene is either completely or partially inhibited. RNAi can also inhibit the function of a CD48 or CD244 RNA, and at least one function can be completely or partially inhibited.

The term “microRNA” refers to single-stranded RNA molecules of about 21-23 nucleotides in length thought to regulate the expression of other genes. miRNAs are encoded by genes that are transcribed from DNA but not translated into protein (non-coding RNA), instead they are processed from primary transcripts known as pri-miRNA to short stem-loop structures called pre-miRNA and finally to functional miRNA. Mature miRNA molecules are complementary to regions in one or more messenger RNA (mRNA) molecules, which they target for degradation.

In other embodiments, CD48 can be influenced through the use of an antagonist of the receptor, a partial antagonist or an antibody that either competes with the natural agonist, blocks the activity or encourages uptake of the CD48 molecule to bind to CD244. In accordance with these embodiments, the CD48 modulating agent can be a nucleic acid sequence, a polypeptide, a protein, a peptide, a fragment thereof, a polynucleotide, an antibody or a small organic molecule. In certain embodiments, a CD48 modulating agent can be an anti-CD48 antibody or fragment thereof capable of binding to CD48 and inhibiting or inducing CD48 ligation. In other embodiments other CD48 modulating agents can include, but are not limited to, a CD48-specific siRNA, RNAi, microRNA or ribozyme. Other CD48 inhibitors and/or antagonists can include anti-CD48 specific antibody fragments (F(ab′)2 or Fab′), single chain Fv, and Fc-fusion protein of CD48 ligands, e.g. Fc fusion proteins of 2B4 or CD2. In some embodiments, an anti-CD48 antibody used herein can be a polyclonal or monoclonal antibody. A “humanized” antibody can be used if needed in order to avoid any potential use incompatibilities (e.g. adverse reactions when introducing the ILC2 cells exposed to such an antibody, if needed).

In certain embodiments, compositions and methods disclosed herein for producing or increasing the production of targeted ILCs can further include at least one cytokine or growth factor. In accordance with these embodiments, a cytokine or growth factor included in compositions herein can stimulate hematopoietic progenitor cell differentiation. In certain embodiments, cytokines or growth factors suitable for cell differentiation as disclosed herein can be, but are not limited to, interferon gamma (IFN-γ), tumor necrosis factor-alpha (TNF-α), interleukin-2 (IL-2), interleukin-12 (IL-12), type I interferons, interferon alpha (INF-α), interferon beta (INF-β), granulocyte-macrophage colony-stimulating factor (GM-CSF), interleukin 1 alpha (IL-1α), interleukin-1 beta (IL-1β), interleukin-2 (IL-2), interleukin-25 (IL-25), interleukin-33 (IL-33), interleukin 3 (IL-3), interleukin-4 (IL-4), interleukin-6 (IL-6), interleukin-7 (IL-7), interleukin-9 (IL-9), interleukin-15 (IL-15), interleukin-18 (IL-18), interleukin-21 (IL-21), interleukin 23 (IL-23), leukemia inhibitory factor (LIF), FMS-like tyrosine kinase 3 ligand (FLT3L), stem cell factor (SCF), or a combination thereof. In some aspects, cytokines or growth factors suitable for use in compositions disclosed herein can be stem cell factor (SCF), interleukin 3 (IL-3), interleukin 7 (IL-7), interleukin 15 (IL-15), interleukin 23 (IL-23), FMS-like tyrosine kinase 3 ligand (FLT3L), or a combination thereof.

In some embodiments, compositions and methods disclosed herein for producing ILCs can further include stroma for expansion or production of the targeted ILC types or in some embodiments, more rapid production of the targeted ILCs. As used herein, the term “stroma” and “stromal cell” can be used interchangeably to refer to an adherent cell that gives rise to cartilage, bone, fat, muscle, and nerve, and is generally present in and isolated from various sources, including, but not limited to, umbilical cord blood, peripheral blood, lymph node/tonsil and other tissues as well as adult bone marrow. In some embodiments, the stroma contemplated herein can be derived from any mammal, including, but not limited to, humans, monkeys, pigs, horses, cows, sheep, dogs, pigs, cats, mice, and rats. In certain embodiments, stroma of use herein can be derived from the mammal to be targeted for ILC therapy such as a human for human therapy or dog for dog therapy, or mixed species, etc. Stromal cells disclosed herein can be obtained by any general methods known in the art. In some embodiments, stroma for use herein can be generated from bone marrow, cell lines, fibroblasts, or other source. In some embodiments, stroma of use herein can be modified to express or over-express CD48 and/or CD244 of use in compositions and methods disclosed herein.

In certain embodiments, CD48 can be overexpressed by the stroma disclosed herein in order to be used to drive production of ILCs and in certain embodiments, ILC2, NCR+ ILC3 and/or LTi-ILC3s. In accordance with these embodiments, CD48 can be overexpressed using methods known to one of skill in the art, including but not limited to, transducing the stroma with a vector expressing CD48. As used herein, the term “vector”, can be an expression vector capable of expressing a protein of interest in a suitable host cell. As used herein, the term “operably linked” refers to a functional linkage between a nucleic acid expression control sequence and a nucleic acid sequence coding for a target protein in such a manner as to allow general functions. The operable linkage to a recombinant vector can be prepared using a genetic recombinant technique well known in the art, and site-specific DNA cleavage and ligation can be achieved using enzymes known in the art. The vector includes plasmid vectors, cosmid vectors, and viral vectors, preferably viral vectors. Examples of the viral vectors can include, but are not limited to, vectors derived from retrovirus such as HIV (Human immunodeficiency virus), MLV (Murine leukemia virus), ASLV (Avian sarcoma/leukosis), SNV (Spleen necrosis virus), RSV (Rous sarcoma virus), and MTV (Mouse mammary tumor virus), adenovirus, adeno-associated virus, and herpes simplex virus, but are not limited thereto.

In certain embodiments, stroma of use and as disclosed herein can be used for driving differentiation of HPCs into ILCs. In some examples, stromal cells disclosed herein can be used as “feeder cells” for hematopoietic progenitors. As used herein, the term “feeder cells” refers to a layer of cells that provide extracellular secretions and/or structure to help HPCs to proliferate during ex vivo expansion. In other embodiments, stromal cells disclosed herein can be irradiated or treated by methods known in the art to reduce or prevent proliferation (e.g. mitomycin C or similar agent). In accordance with these embodiments, irradiation can lead to the stromal cells becoming growth arrested while still viable and producing increased concentrations of hematopoietic growth factors/cytokines of use in compositions and methods disclosed herein. In some embodiments, stroma overexpressing CD48 as disclosed herein and contemplated of use herein can be irradiated for partial or complete growth arrested viable cells. In other embodiments, the CD48 overexpressed in the stroma can be a CD48 ligand.

In other embodiments, other molecules can be expressed or over-expressed on stroma of use herein. In accordance with these embodiments, other SLAM family molecules CD84, CD319 NTB-A and CD229) could be over expressed on stroma and used to induce ILC differentiation. In certain embodiments, overexpression of SAP or EAT-2 can be used to overexpress ILC2. In yet other embodiments, knock down of SHIP, SHP1 and SHP2 can be used to increase ILC2.

In certain embodiments, ILC2s can be generated from HPCs or iPSCs disclosed herein. In some embodiments, ILC2 cells (and/or ILC3 cells (including, for example, NCR+ ILC3 and LTi-ILC3) can be generated from HPCs expressing CD48 in the presence of the compositions disclosed herein that can include a CD48 ligand, a CD48 agonist, a CD48 antagonist, or a combination thereof. In certain embodiments the CD48+ cells can be CD52⁻ cells not expressing CD52 and/or Lin⁻ cells. In other aspects, a CD48 modulating agent as disclosed herein can be added to the cell culture medium after HPC harvesting and as applicable, preparation or enrichment of progenitor cells of ILCs. In other aspects, a CD48 modulating agent can be added to the cell culture medium after 1 day, 2 days, 3 days, 4 days or up to 20 days or more following HPC harvest and/or isolation or enrichment. In some embodiments, a CD48 modulating agent or binding or blocking agent can be added before, after or at the same time as growth factors, chemokines and/or cytokines, as described herein, to induce differentiation of HPCs into ILCs. In other embodiments, a CD48 modulating agent can be added to the stroma prior to seeding the HPCs on the stroma. In yet other embodiments, a CD48 modulating agent can be CD48-overexpressing stroma.

Cells of use in methods and compositions disclosed herein can be cultured in culture medium that is established in the art and commercially available from the American Type Culture Collection (ATCC). For example, media can include, but are not limited to, Dulbecco’s Modified Eagle’s Medium (DMEM), DMEM F12 medium, Eagle’s Minimum Essential Medium, F-12K medium, Iscove’s Modified Dulbecco’s Medium, RPMI-1640 medium, serum-free media, media with serum and/or supplemented media optimal to expand HPCs in culture. It is within the skill of one in the art to modify or modulate concentrations of media and/or media supplements as needed for the cell type used or cell source.

In certain embodiments, additional supplements can also be used to supply the cells with trace elements for improved growth and expansion. Such supplements can include, but are not limited to, insulin, transferrin, sodium selenium, and combinations thereof. These components can be included in any known acceptable form. In some embodiments, these agents can be in a salt solution including, but not limited to, Hanks’ Balanced Salt Solution® (HBSS), Earle’s Salt Solution®, antioxidant supplements, MCDB-201® supplements, phosphate buffered saline (PBS), N-2-hydroxyethylpiperazine-N′-ethanesulfonic acid (HEPES), nicotinamide, ascorbic acid and/or ascorbic acid-2-phosphate, as well as additional amino acids. In some examples, amino acids for use herein can include, but are not limited to, L-alanine, L-arginine, L-aspartic acid, L-asparagine, L-cysteine, L-cystine, L-glutamic acid, L-glutamine, L-glycine, L-histidine, L-inositol, L-isoleucine, L-leucine, L-lysine, L-methionine, L-phenylalanine, L-proline, L-serine, L-threonine, L-tryptophan, L-tyrosine, and L-valine.

In other embodiments, as needed, antibiotics can be used in cell cultures to mitigate or eliminate bacterial, mycoplasmal, and fungal contamination. Typically, antibiotics or anti-mycotic compounds used are mixtures of penicillin/streptomycin, but can also include, but are not limited to, amphotericin (Fungizone®), ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin.

Hormones can also be used in cell cultures and include, but are not limited to, D-aldosterone, diethylstilbestrol (DES), dexamethasone, β-estradiol, hydrocortisone, insulin, prolactin, progesterone, somatostatin/human growth hormone (HGH), thyrotropin, thyroxine, and L-thyronine. β-mercaptoethanol and other hormones contemplated herein.

Lipids and lipid carriers can also be used to supplement cell culture media, depending on the type of cell and the fate of the differentiated cell. Such lipids and carriers can include, but are not limited to cyclodextrin (a, β, γ), cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, among others.

In some embodiments, cells of the present disclosure in culture can be maintained either in suspension or attached to a solid support, such as a coated plate or where extracellular matrix components and synthetic or biopolymers are included. Cells can also be supplemented with additional factors that encourage their attachment to a solid support including, but not limited to, type I, type II, and type IV collagen, concanavalin A, chondroitin sulfate, fibronectin, “superfibronectin” and/or fibronectin-like polymers, gelatin, laminin, poly-D and poly-L-lysine, Matrigel™, thrombospondin, and/or vitronectin. In certain embodiments, multi-well plates can be used (e.g. G-Rex culture plates).

The cells described herein can be selected based on the markers (gene and/or protein) described herein. Accordingly, positive selection methods can be used, either alone or together with the methods described above, to identify and/or isolate the cells of the invention. Methods of positive selection can include visual selection, using microscopy and/or other means of detection, including, but not limited to, immunoblotting, immunofluorescence, and/or enzyme-linked immunosorbent assay. Other methods of positive selection can also include, but are not limited to, additional selective culture techniques (e.g., variable cell densities or amounts of CO₂), flow cytometry, RT-PCR, and/or microchip-based methods of cell separation. Negative selection methods can also be used.

In certain embodiments, growth factors, chemokines and/or cytokines, as described herein, can be provided to a cell culture to assist in inducing differentiation to a desired ILC type. For example, IL-3, IL-2, IL-7, IL-15, IL-25, IL-33, and other factors, such as stem cell factor and FLT-3L (that will be encompassed by the term cytokine herein) can differentiate HSC to NK, ILC2, or ILC3 cells or immediate progenitor cells thereof. In other embodiments, providing stem cell factor (SCF), interleukin 3 (IL-3), interleukin 7 (IL-7), interleukin 15 (IL-15), interleukin 23 (IL-23), FMS-like tyrosine kinase 3 ligand (FLT3L), or a combination thereof to a composition disclosed herein can assist in differentiating HSC to ILC2s or ILC3 s (including, for example, NCR+ ILC3 and LTi-ILC3).

In certain embodiments, methods disclosed herein can differentiate HSC to ILC2 cells. In some embodiments, the methods of generating ILC2 and/or ILC3s (including, for example, NCR+ ILC3 and LTi-ILC3) disclosed herein can yield ILC2 cell expressing CD11a, CD117, or a combination thereof or ILC3 cells expressing CD117, CD336 or other markers of ILC3s (including, for example, NCR⁺ ILC3 and LTi-ILC3).

In some embodiments, methods of generating ILC2 cells disclosed herein can yield a enriched population of ILC2 cells. In some aspects, methods of generating ILC2 cells disclosed herein can yield a population of ILC cells at is about 50%, to about 60%, to about 70%, to about 80%, to about 99% homogenous for ILC2 cells. In other embodiments, compositions and methods disclosed herein to produce ILC2s or ILC3s (including, for example, NCR+ ILC3 and LTi-ILC3) can be a mixture of cells but enriched for these targeted cells and, optionally, be further isolated for uses disclosed herein.

In other embodiments, methods of generating ILC2 cells disclosed herein can yield a population of ILC2 cells that is about 40%, to about 50%, to about 60% to about 70% to about 80%, to about 90%, to about 99% enriched in IL-2 cells. In some embodiments, methods of generating ILC2 cells disclosed herein can yield a population of ILC2 cells that is at least about 70% to about 80% of the cell population. In other embodiments, compositions and methods disclosed herein to produce ILC2s or ILC3s (including, for example, NCR-ILC3 and LTi-ILC3) can be a mixture of cells but enriched for these targeted cells and, optionally, be further isolated for uses disclosed herein.

Certain methods disclosed herein provide methods for treating one or more immune-mediated conditions or immune-mediated condition or disease in a subject by administering a composition including, but not limited to, ILC2 or ILC3s (including, for example, NCR+ ILC3 and LTi-ILC3) cells prepared by methods disclosed herein. Non-limiting examples of such immune-mediated diseases or conditions include, but are not limited to, graft versus host disease (GvHD), inflammatory bowel diseases (IBD), Crohn’s disease Type-1 Diabetes, renal disease or renal condition or injury, psoriasis, asthma, allergies, rheumatoid arthritis, ankylosing spondylitis, cardiac conditions or cardiovascular disease, psoriasis, psoriatic arthritis, Behcet’s disease, arthritis, viral infections (e.g., DNA viruses (Adenoviruses, Herpesviruses (e.g., Herpes simplex, type 1, Herpes simplex, type 2, Varicella-zoster virus, Epstein-barr virus, Human cytomegalovirus, Human herpesvirus, type 8), Papillomaviridae (e.g., Human papillomavirus), Poxviruses (e.g., Smallpox), Parvoviruses (e.g., Human bocavirus, Parvovirus B19), Hepadnaviridae (e.g., Hepatitis B virus) and/or Reoviruses (e.g., Rotavirus)), RNA viruses (Picornaviruses (e.g., coxsackievirus, hepatitis A virus, poliovirus, rhinovirus), Togaviruses (Rubella virus), Orthomyxoviruses (e.g., Influenza virus), and/or Rhabdoviruses (e.g., Rabies virus)), or reverse transcribing viruses (including, but not limited to, Retroviruses and Hepadnaviruses, Retroviridae (human immunodeficiency virus (HIV)), Metaviridae, Pseudoviridae, Caulimoviridae, Hepadnaviridae)). Also contemplated in immune-mediated conditions are certain types of cancers. Such cancers include, but are not limited to, cancer (including, but not limited to, carcinoma (e.g., breast, prostate, lung, pancreas, liver (e.g., hepatocarcinoma) or colon cancer), sarcoma (e.g., bone, cartilage, neuronal or fat (e.g., liposarcoma) cancers), lymphoma, leukemia (blood type cancers), blastomas (e.g., hepatoblastoma). In some embodiments, the condition in need of treatment in a subject is GvHD or Type-1 diabetes, IBD or cardiovascular disease or renal injury or renal disease or the like.

The term “subject” as used herein can refer to any mammal, including but not limited to, a non-human primate (for example, a monkey or great ape), livestock or pets such as a cow, a pig, a cat, a dog, a rat, a mouse, a horse, a goat, a rabbit, a sheep, a hamster, a guinea pig) or other subject. In some embodiments, the mammalian subject is a human such as an adult, a young child, adolescent, toddler, infant or fetus.

For the purposes described herein, either autologous, allogeneic or xenogeneic ILC2s or ILC3 (including, for example, NCR+ ILC3 and LTi-ILC3s) of the present disclosure can be administered to a subject. In accordance with these embodiments the ILCs can be either in undifferentiated, partially differentiated or fully differentiated forms, genetically altered or unaltered, introduced by direct injection to a tissue site, by infusion through a portal vein, in a bolus delivered to an organ, administered systemically, on or around the surface of an acceptable matrix, encapsulated or in combination with a pharmaceutically acceptable carrier.

In some embodiments, ILC2 or ILC3s (including, for example, NCR+ ILC3 and LTi-ILC3) cells produced by compositions and methods disclosed herein can be prepared for administering to a subject by any suitable method known in the art. In some embodiments, cells can be administered to a subject by localized or systemic injection. In some embodiments, ILC2 and/or ILC3s (including, for example, NCR+ ILC3 and LTi-ILC3) cell preparations can be administered by comparable methods to bone marrow implantation, such as through a renal artery or similar. In other embodiments, ILC and/or ILC3s (including, for example, NCR+ ILC3 and LTi-ILC3) cell preparations can be introduced directly to a site of interest such as an infection or other area in need of such a treatment.

In some embodiments, the number of cells implanted into a subject can be a therapeutically effective number or amount. As used herein, a “therapeutically effective amount” can refer to the number of transplanted cells that have a treatment effect for a particular injury, disease or condition for which treatment is sought. For example, where the treatment is for tissue injury, implantation of a therapeutically effective amount of cells can typically produce a reduction in the severity of the symptoms associated with the injury. Persons or health professionals of skill in the art will understand how to determine proper cell dosages.

In other embodiments, cells of the present disclosure and their differentiated progeny can be induced to proliferate and/or differentiate in vivo if desired, by administering to the subject, growth factor(s), cytokine(s) or pharmaceutical composition(s) that will induce proliferation and differentiation of the cells. These growth factor(s), cytokine(s) or pharmaceutical composition(s) include any growth factor, cytokine or pharmaceutical composition known in the art, including the growth factors and cytokines described herein for in vitro proliferation and differentiation.

Exogenous factors (e.g., cytokines, differentiation factors and other factors) can be administered prior to, after or concomitantly with the cells of the invention introduced to the subject. For example, a form of concomitant administration could include combining a factor of interest in the culture media and/or pharmaceutically acceptable carrier prior to administration. Doses for administrations are variable and can include an initial administration followed by subsequent administrations; and can be ascertained by the skilled artisan and from the present disclosure.

In some embodiments, the quantity ILC2 or ILC3s (including, for example, NCR+ ILC3 and LTi-ILC3) of the present disclosure to be administered can be optimized to achieve an optimal effect in a subject. Different scenarios can require optimization of the number of cells injected into a tissue of interest. For example, the quantity of cells to be administered can vary for the subject being treated. In one embodiment, between 10⁴ to 10⁸, or 10⁵ to 10⁷, or around 10⁷ cells or more cells can be administered in a single bolus or in multiple boluses for optimal effect. However, the precise determination of what would be considered an effective dose can be based on factors individual to each patient, including their size, age, degree of tissue injury/damage, and length of time from when the injury occurred. Dosages can be readily ascertained by those skilled in the art from this disclosure and the knowledge in the art and in consideration of bone marrow transplantation doses or other cellular implant doses.

The pharmaceutical formulations suitable for injection include sterile aqueous solutions and dispersions. The carrier can be a solvent or dispersing medium containing, for example, water, saline, phosphate buffered saline, polyol (for example, glycerol, propylene glycol, liquid polyethylene glycol, and the like) and suitable mixtures thereof.

In some embodiments, certain additives which enhance the stability, sterility, and isotonicity of the compositions, including antimicrobial preservatives, antioxidants, chelating agents, and buffers, can be added to the contemplated compositions herein. In some embodiments, antibacterial and antifungal agents can be added to reduce contamination of cultures for administration, for example, parabens, chlorobutanol, phenol, sorbic acid, and the like.

Sterile injectable solutions can be prepared by incorporating the cells utilized in practicing the present disclosure in the required amount of the appropriate solvent with certain amounts of the other ingredients, as desired. Examples of compositions including the ILC cells of the invention can include liquid preparations for administration, including suspensions. Such compositions can be in admixture with a suitable carrier, diluent, or excipient such as sterile water, physiological saline, glucose, dextrose, or the like.

Compositions of the present invention can be provided as liquid preparations, e.g., isotonic aqueous solutions, suspensions, emulsions or viscous compositions, which can be buffered to a selected pH. The choice of suitable carriers and other additives can depend on the route of administration and the nature of the particular dosage form, e.g., liquid dosage form (e.g., whether the composition is to be formulated into a solution, a suspension, gel or another liquid form, such as a time release form or liquid-filled form). Solutions, suspensions and gels normally contain a major amount of water (e.g., purified, sterilized water) in addition to the cells. Minor amounts of other ingredients such as pH adjusters (e.g., a base such as NaOH), emulsifiers or dispersing agents, buffering agents, preservatives, wetting agents and jelling agents (e.g., methylcellulose), can also be present. The compositions can be isotonic, i.e., they can have the same osmotic pressure as blood and lacrimal fluid. In other embodiments, agents can be provided to reduce cell lysing or other adverse effect on the cells for delivery to a subject.

In some embodiments, desired isotonicity of the cell compositions of the present disclosure can be accomplished using sodium chloride, or other pharmaceutically acceptable agents such as dextrose, boric acid, sodium tartrate, propylene glycol or other inorganic or organic solutes. Viscosity of the compositions, if desired, can be maintained at the selected level using a pharmaceutically acceptable thickening agent. Methylcellulose is readily and economically available and is easy to work with. Other suitable thickening agents include, for example, xanthan gum, carboxymethyl cellulose, hydroxypropyl cellulose, carbomer, and the like. The concentration of the thickener will depend upon the agent selected. The point is to use an amount, which will achieve the selected viscosity. Viscous compositions are normally prepared from solutions by the addition of such thickening agents.

A pharmaceutically acceptable preservative or cell stabilizer can be employed to increase the life of the compositions. If preservatives are used, it is well within the purview of the skilled artisan to select compositions that will not affect the viability or efficacy of the cells as described herein.

Compositions can be administered in dosages and by techniques well known to those skilled in the medical and veterinary arts taking into consideration such factors as the age, sex, weight, and condition of the particular patient, and the composition form used for administration (e.g., solid vs. liquid). Dosages for humans or other mammals can be determined without undue experimentation by the skilled artisan, from this disclosure, and the knowledge in the art.

In some embodiments, kits are contemplated of use to generate the ILC2 cells disclosed herein. In certain embodiments, kits can include a hematopoietic progenitor cell expressing CD48 and CD244 and at least one of a CD48 ligand or a CD244 agonist and at least one container. In other embodiments, the progenitor cells are CD48 positive and CD52 negative cells. In other embodiments, a kit can further include least one cytokine, growth factor, stroma, cell culture medium, buffers, or a combination thereof. In certain embodiments, kits can include ILC2s and/or ILC3s (including, for example, NCR+ ILC3 and LTi-ILC3) and at least one container. In other embodiments, a kit can further include an insert with instruction to generate ILC2 and/or ILC3s (including, for example, NCR+ ILC3 and LTi-ILC3) from HPCs according to the methods disclosed herein.

The present disclosure also provides kits for use in treating or alleviating a targeted disease or condition treatable by use of ILCs, such as an immune-mediated or immunocompromised condition or disease disclosed herein. In some embodiments, the kit can include instructions for use in accordance with any of the methods described herein. Instructions found in a kit can include a description of administration of the ILC2 and/or ILC3 cell-containing composition, and optionally the second therapeutic agent, to treat, delay the onset, or alleviate a target disease as those described herein. The kit can further include a description of selecting an individual suitable for treatment based on identifying whether that individual has the target disease or condition, e.g., applying the diagnostic method as described herein and/or identifying symptoms in the subject. In yet other embodiments, the instructions can include a description for administering an antibody to a subject at risk of developing a disease or condition disclosed herein.

In some embodiments, instructions relating to the use of an ILC2 cell containing composition generally include information including but not limited to, dosage such as number of cells, dosing schedule, and route of administration for the intended treatment. Containers of kits can include unit dosing or bulk packages (e.g., multi-dose packages) or sub-unit doses. Kits can further include a delivery device such as a syringe, implant device or cellular delivery device. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease, such as cancer or immune disorders (e.g., autoimmune disease). Instructions can be provided for practicing any of the methods described herein.

In some embodiments, kits can be in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit can have a sterile access port (for example the container can be an intravenous solution bag or a vial having a stopper pierceable by a hypodermic injection needle). The container can also have a sterile access port (for example the container can be an intravenous solution bag or a vial having a stopper pierceable by a hypodermic injection needle). Kits contemplated herein can contain at least one active agent in the composition such as progenitor cells disclosed herein or at least one of ILC2s and ILC3s (including, for example, NCR+ ILC3 and LTi-ILC3) as described herein.

Kits can optionally provide additional components such as buffers and interpretive information. Normally, the kit includes a container and a label or package insert(s) on or associated with the container. In some embodiments, the invention provides articles of manufacture comprising contents of the kits described above.

### EXAMPLES

The following examples are included to illustrate certain embodiments and are not considered limiting to the instant disclosure. It should be appreciated by those of skill in the art that the techniques disclosed in the examples which follow represent techniques discovered to function in the practice of the claimed methods, compositions and apparatus. However, those of skill in the art should, in light of the present disclosure, appreciate that changes can be made in some embodiments and examples which are disclosed and still obtain a like or similar result without departing from the spirit and scope of the invention.

### Example 1

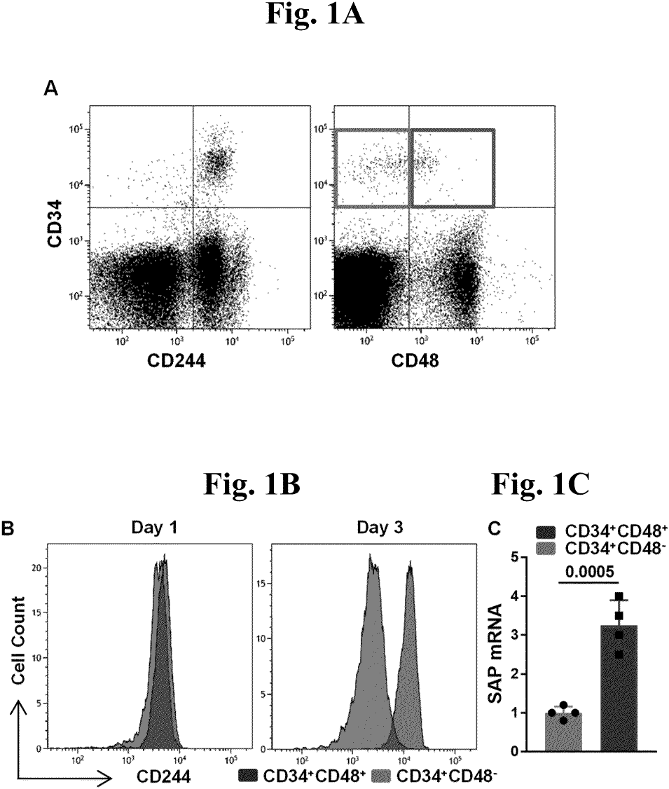

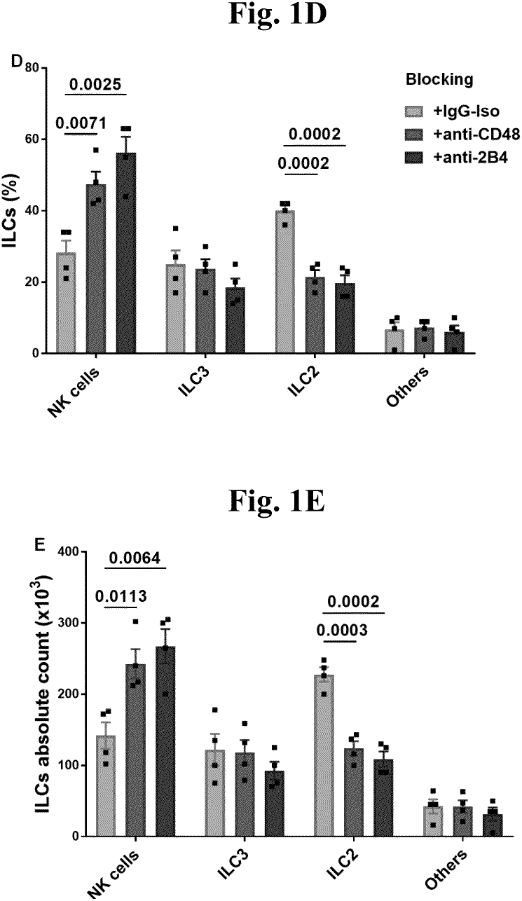

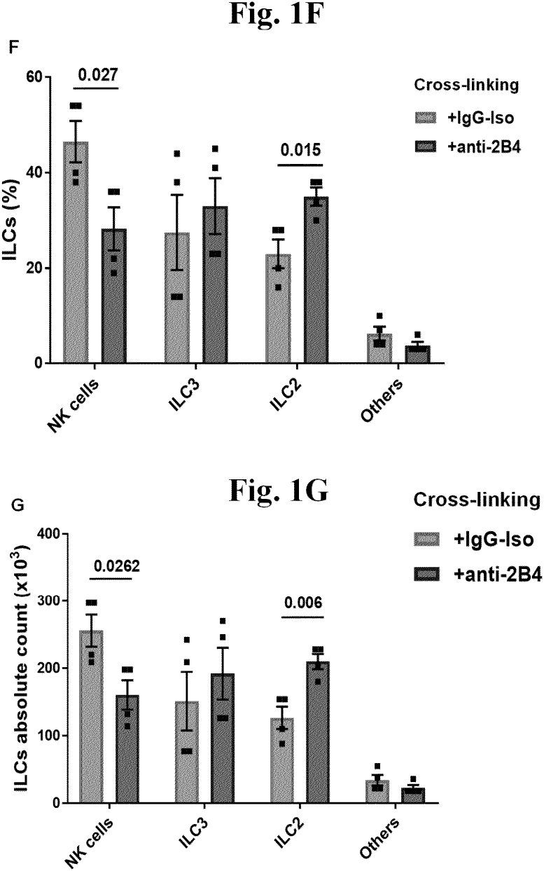

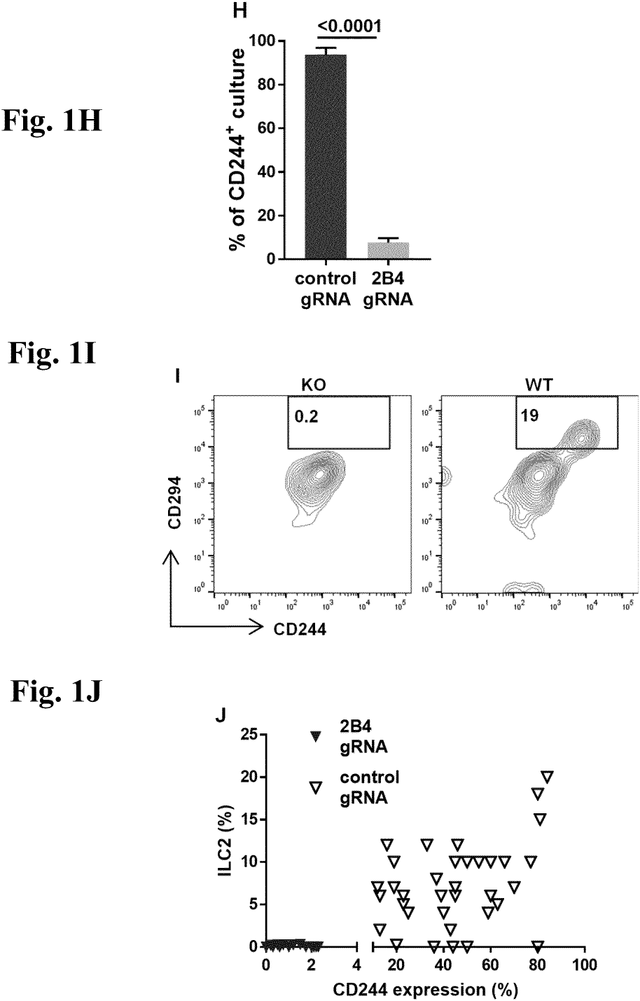

In one exemplary method, compositions including harvested HPC progenitor cells were used in culture in order to induce increased production of or differentiation of ILCs. As illustrated in FIGS. 1A-1J exemplary methods of enhancing ILC2 Development by CD244 activation are demonstrated. FIG. 1A illustrates representative flow cytometry data depicting freshly isolated UCB-derived CD34⁺ HSCs stained for CD244 and CD48. FIG. 1B illustrates representative flow cytometry data depicting cell counts representing CD244 in CD34⁺CD48⁻ cells and in CD34⁺CD48⁺ cells for day 1 and day 3 UCB-derived CD34⁺ HSCs. FIG. 1C illustrates a bar graph representing SAP mRNA expression in day 5 CD34⁺CD48⁻ and CD34⁺CD48⁺ cells. FIGS. 1D and 1E illustrate bar graphs depicting differentiating CD34⁺α4β7⁺CD48⁺ cells stained for ILCs after CD244 signaling was blocked by the addition of an anti-CD244 or anti-CD48 blocking antibody during differentiation where FIG. 1D depicts the percentage of CD94⁺ NK, CD294⁺ ILC2, and CD117⁺ ILC3 cells and FIG. 1E depicts the absolute number of CD94⁺ NK, CD294⁺ ILC2 and CD117⁺ ILC3 cells. FIGS. 1F and 1G illustrate bar graphs depicting differentiating CD34⁺α4β7⁺CD48⁺ cells stained for ILCs after CD244 signaling was activated by the addition of a cross-linking antibody during differentiation where FIG. 1F depicts the percentage of CD94⁺ NK, CD294⁺ILC2, and CD117⁺ ILC3 cells and FIG. 1G depicts the absolute number of CD94⁺ NK, CD294⁺ILC2 and CD117⁺ ILC3 cells. FIG. 1H illustrates a bar graph depicting CRISPR-Cas9 knockdown of CD244 expression in CD34⁺α4β7⁺CD48⁺ progenitor cells transfected with control or CD244 gRNA. FIG. 1I illustrates representative flow cytometry data of CD244⁺ cultures for cells transfected with control or CD244 gRNA staining for ILC2 (CD294). FIG. 1J illustrates a dot plot depicting representative generation of ILC2s from cultures that express (control gRNA) or lack (CD244 gRNA) CD244.

### Example 2

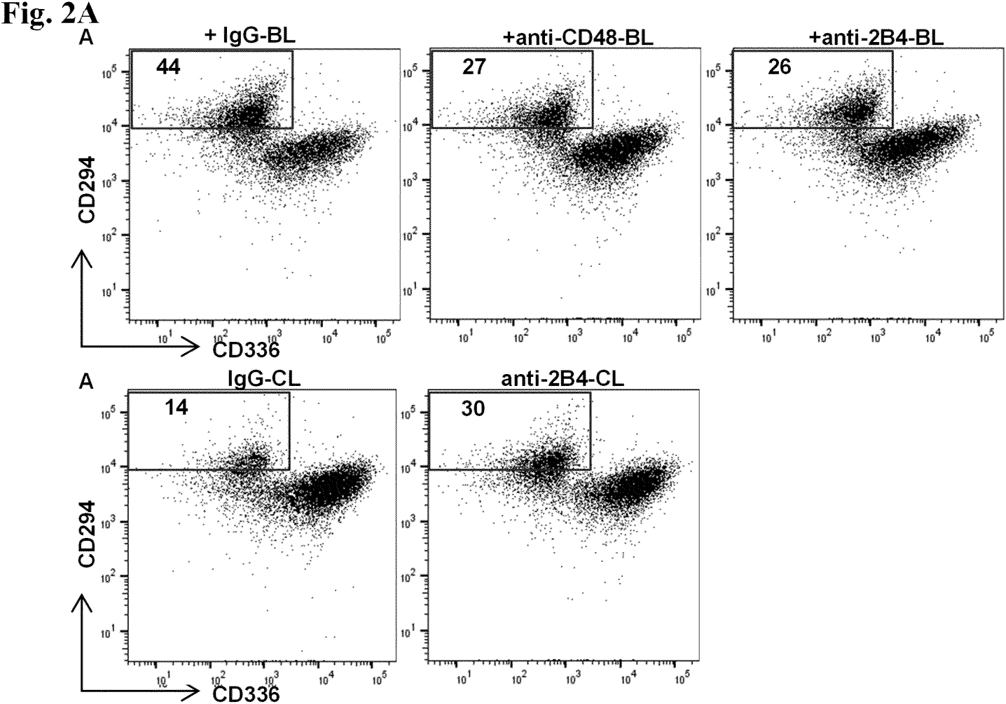

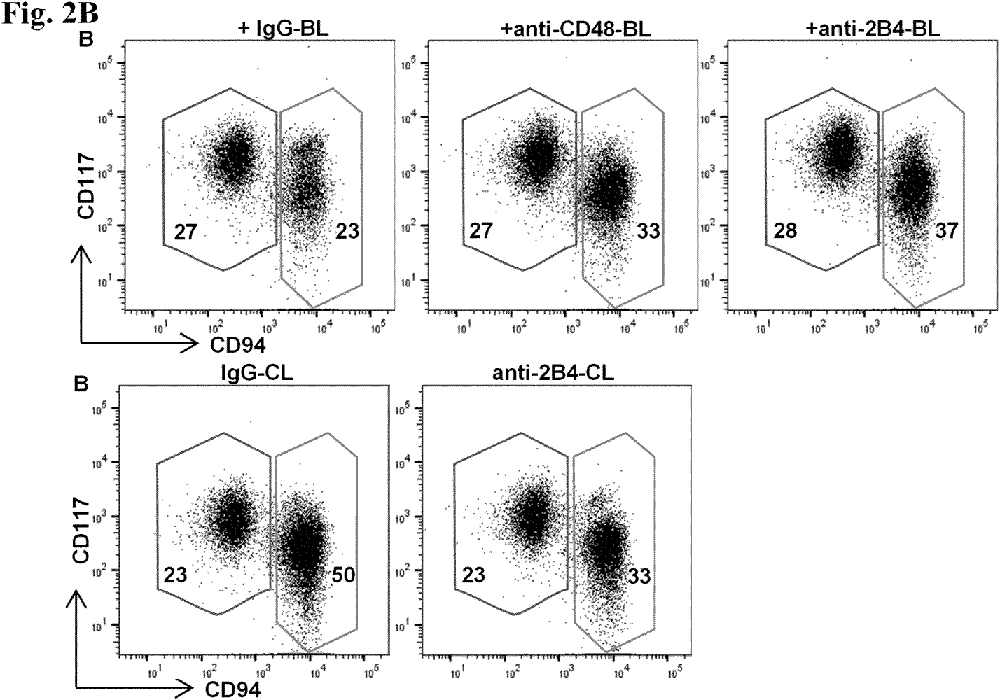

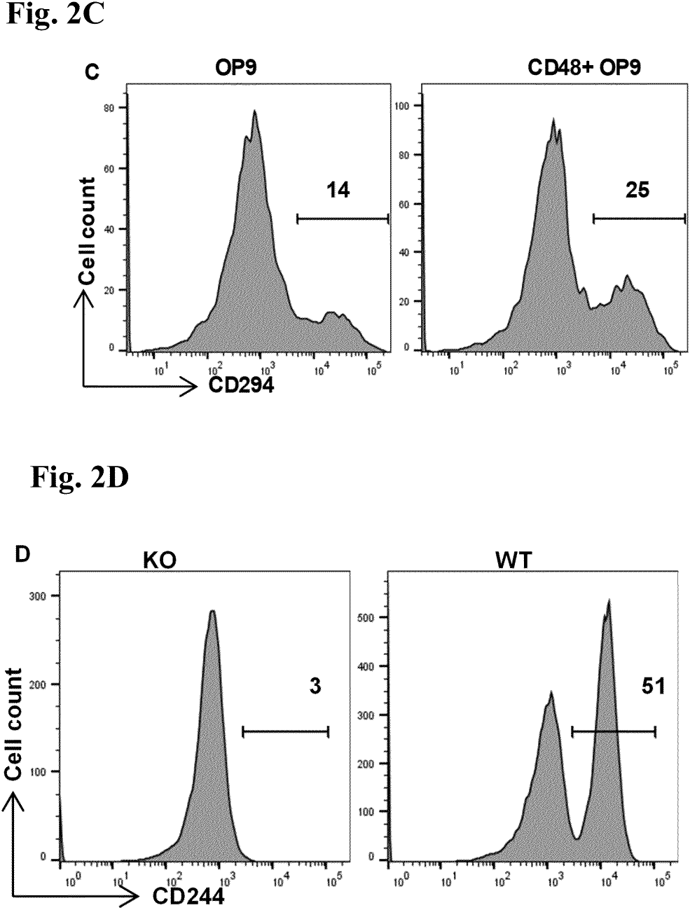

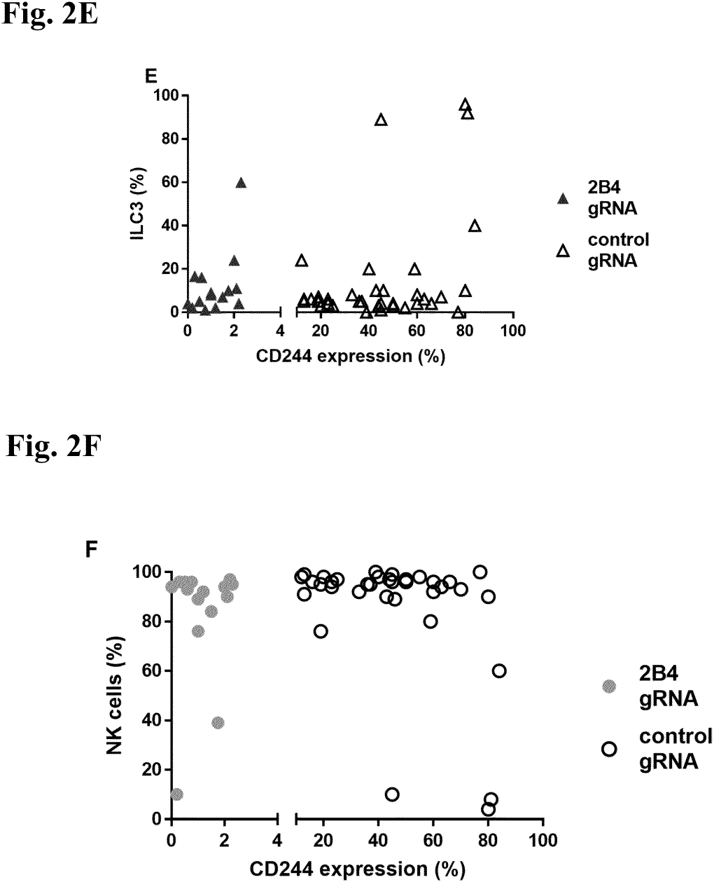

In other exemplary methods, additional studies were performed in order to evaluate use of CD48 targeting agents to induce differentiation of HSCs into ILCs. As demonstrated in this example, FIGS. 2A-2F represent exemplary methods of enhancing ILC2 Development by CD244 activation. FIG. 2A illustrates representative flow cytometry data depicting CD34⁺α4β7⁺CD48⁺CD52⁺ differentiating cultures treated with anti-CD244 and anti-CD48 blocking antibodies or isotype IgG. FIG. 2B illustrates representative flow cytometry data depicting CD34⁺α4β7⁺CD48⁺CD52⁺ differentiating cultures treated with a CD244 cross-linking antibody. FIG. 2C illustrates representative flow cytometry data depicting CD294 staining for CD34⁺α4β7⁺CD48⁺ cultures on a layer of irradiated CD48 expressing OP9 or control OP9 stromal cells. FIG. 2D illustrates representative flow cytometry data depicting CD244 staining for CD34⁺α4β7⁺CD48⁺ cultures transfected with control gRNA (wild type) or CD244 gRNA. FIGS. 2E and 2F illustrate dot plots depicting generation of ILC3s (FIG. 2E) and NK cells (FIG. 2F) from cultures that express (control gRNA) or lack (2B4 gRNA) CD244.

It was observed in these studies that the majority of freshly isolated UCB-derived CD34⁺ HSCs expressed CD244, while only a proportion stained for CD48 (FIG. 1A). While CD244 surface density on freshly isolated CD34⁺CD48⁻ HSCs was similar to CD34⁺CD48⁺ HSCs, it was significantly upregulated on the latter progenitors after three days of culture (FIG. 1B). It was next determined whether CD244 signaling influences ILC differentiation from CD34⁺α4β7⁺CD48⁺ cells. Indeed, SLAM-associated protein (SAP), known as an adapter protein for CD244 signaling was significantly upregulated within CD34⁺α4β7⁺CD48⁺ cell population, indicating activation of CD244 signaling (FIG. 1C). During development, the addition of anti-CD244 or anti-CD48 blocking antibody significantly abrogated ILC2 differentiation, while the proportion and absolute number of NK cells were increased (FIGS. 1D and 1E and FIGS. 2A and 2B). Conversely, activation of CD244 signaling (using an agonist cross-linking antibody) increased ILC2 differentiation at the expense of NK cells (FIGS. 1F and 1G and FIGS. 2A and 2B). Moreover, culturing CD34⁺α4β7⁺CD48⁺ progenitors on a layer of irradiated CD48 expressing OP9 (vs. control OP9 stromal cells) showed an enhanced ILC2 generation (FIG. 2C). In this example, the agonist cross-linking antibody includes, but is not limited to, a commercially available monoclonal antibody raised in mouse, IgG1 kappa isotype and clone eBioC1.7 (C1.7). This antibody is a functional grade, affinity chromatography purified monoclonal CD244 antibody (e.g. Thermofisher, CD244 Antibody, Functional Grade (16-5838-85))

To further confirm this, CRISPR-Cas9 was used to knockout CD244 in CD34⁺α4β7⁺CD48⁺progenitors followed by single cell culture on irradiated OP9 feeders. Using this approach CD244 was lost in 92% of the single cell cultures, whereas 94% of the single cell cultures containing control gRNA expressed CD244 (FIG. 1H and FIG. 2D). Impressively, in the absence of CD244 there was a complete loss of ILC2 development (FIGS. 1I and 1J). In contrast, NK cells and ILC3s differentiated from CD244 knockout progenitors (FIGS. 2E and 2F). As above, two separate ILC2 populations were identified based on CD11a and CD117 expression and both ILC2 subsets increased proportionally with CD244 activation, suggesting that CD244 signaling acts upstream of ILC2 subtype specification. In summary, these data demonstrated that CD244 signaling influences ILC differentiation by modulating the development of CD34⁺α4β7⁺CD48⁺ progenitors into ILC2 cells.

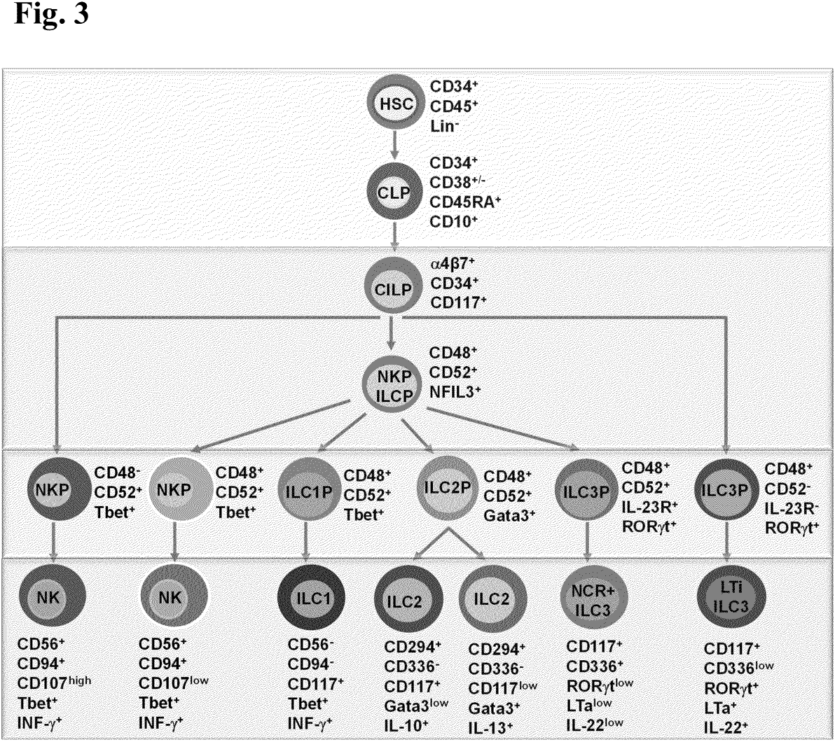

These exemplary methods demonstrate how CD48, via 2B4 receptor signaling, enhances the development of mature ILC2 from HSCs (FIG. 3). Furthermore, exemplary methods described herein demonstrate that the use of a hematopoietic progenitor cell expressing CD48 in addition to a CD48 ligand, a CD48 agonist or a CD48 antagonist can produce a relatively homogenous population of viable ILC2 cells.

### Example 3

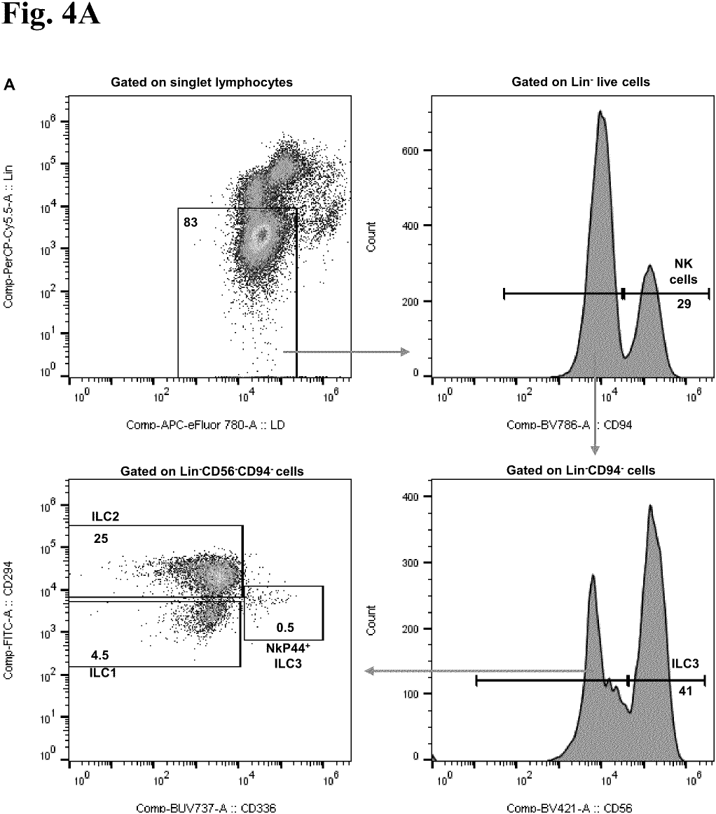

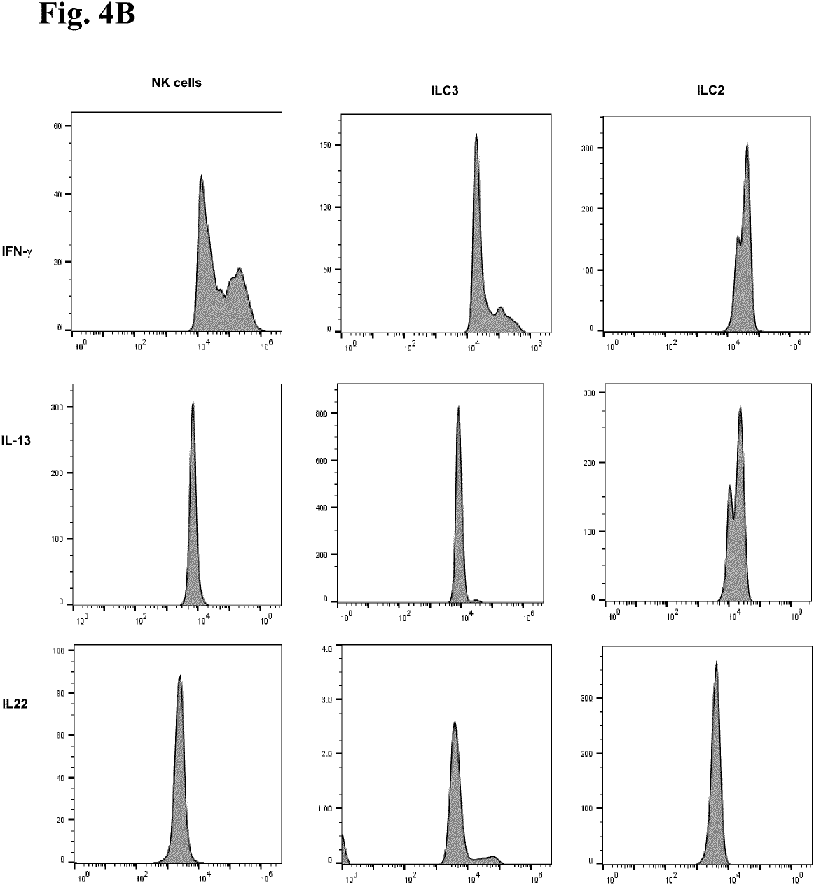

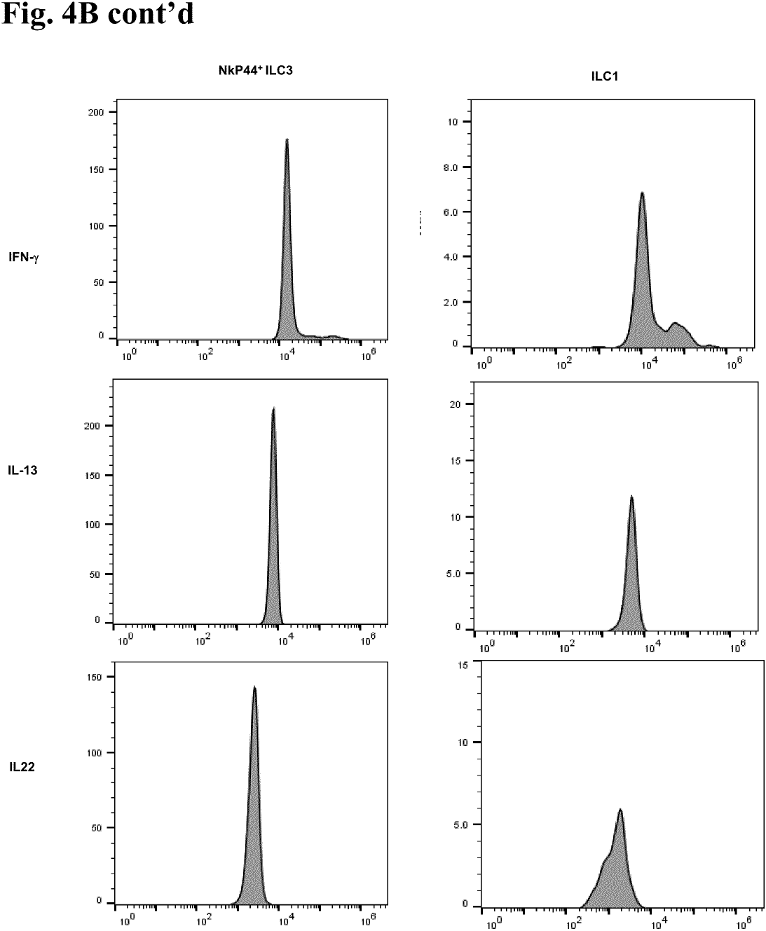

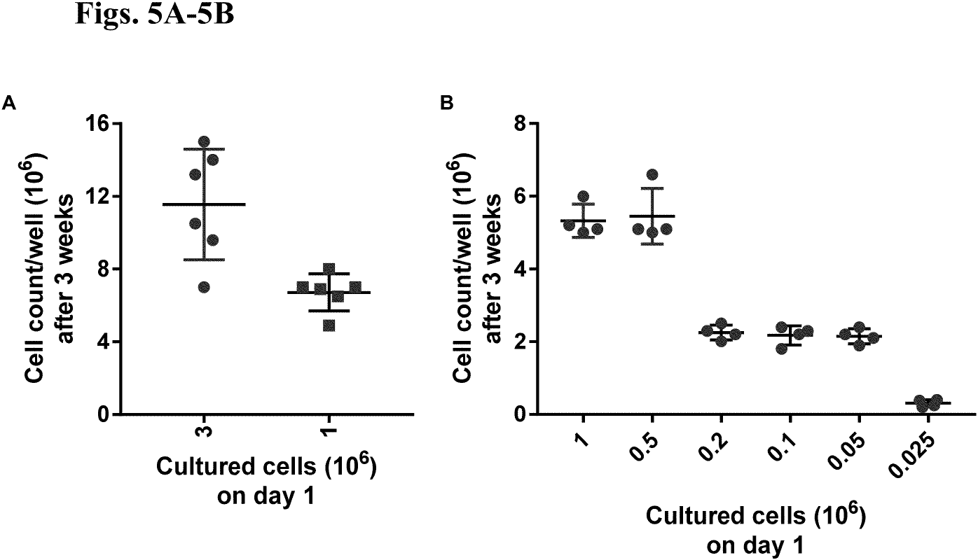

In another exemplary method, CD34+ HSCs were positively enriched from cord blood unit using in this example, a MACS CD34+ enrichment kit (Milteny). The cells were suspended (5×10⁴ cells/ml) in cell culture media (e.g. Stemspan II, Stemcell) supplemented with 1% penicillin + streptomycin, stem cell factor (SCF, 100 ng/ml, R&D), FMS-like tyrosine kinase 3 (Flt3L, 100 ng/ml, Stemcell), thrombopoietin (TPO, 50 ng/ml, R&D) and low-density lipoprotein (LDL, 10ug/ml, Stemcell) and cultured in 24 well plates for 5 days of expansion. On average, ~2×10⁶ CD34+α4β7+ progenitors were sorted from day 5 expanded CD34+ HSCs using FACS and further expanded for 2 days and this resulted in a total of 10×106 (5-fold increase) CD34+α4β7+ progenitors from a single cord unit. Cells were then cultured for 21 days of differentiation in BO media, supplemented with SCF (20 ng/ml, R&D Systems), IL-3 (5 ng/ml, Stemcell), IL-7 (20 ng/ml, R&D), IL-15 (10 ng/ml, NIH), IL-23 (10 ng/ml, R&D) and Flt3L (10 ng/ml, Stemcell) using 24- or 6- wells G-Rex multi-well cell culture plates. It was demonstrated that CD34+α4β7+ progenitors are composed of different subsets that give rise non-ILCs, NK cells and ILCs. For instance, CD34+α4β7+Lin-CD48-CD52- subsets differentiate into Lin+ non-ILCs. Whereas, CD34+α4β7+Lin-CD48+CD52+ gives rise to multiple ILC types (ILC1, -2, -3 cells), while CD34+α4p7+Lin-CD48-CD52+ and CD34+α4β7+Lin-CD48+CD52- progenitors specifically give rise to NK cells and LTi-like ILC3s, respectively. A representative donor is indicated in FIG. 4A demonstrating that 17% of the differentiated cells from CD34+α4β7+ progenitors in G-Rex multi-well cell culture plates are Lin+ cells, whereas 83% of the progeny are NK cells and ILCs (n=6). Of the Lin- cells, NK cells, ILC1, ILC2 and ILC3s contributed to 29%, 4.5%, 25% and 41.5%, respectively (FIG. 4A, n=6). Further confirmation of cell identity was established by cytokine production, showing IFN-y+ NK cells and ILC1s, IL-13+ ILC2s and IL-22+ ILC3s (FIG. 4B). G-Rex multi-well cell culture system resulted in hundreds of millions of NK cells and ILCs (FIGS. 5 and Table 1).

FIGS. 4A-4B represent staining strategy of ILCs differentiated in G-Rex multi-well cell culture plate. UCB-derived CD34+ HSCs were expanded for 5 days, CD34+α4β7+ progenitors were sorted by FACS and expanded for 2 more-days. ILCs were differentiated from CD34+α4β7+ progenitors in G-Rex multi-well cell culture plate for 21 days in the presence of cytokines including IL-3 (only for week 1), IL-7, IL-15, IL-23, SCF and FLT3L and stained for surface receptors and intracellular cytokines. Antibodies for lineage markers (containing CD1a, CD3, CD4, CD5, CD11c, CD14, CD19, CD34, TCRαβ, TCRɣδ, FcεRI, CD123, and CD303) were used to exclude any possible lineage contamination, while viability dye was used to exclude dead cells. (A) The percentage of Lin-live cells (NK cells+ILCs) were shown (top left plot) and ILCs were identified using surface receptors for NK cells, ILC1, ILC2, ILC3 and NKp44+ ILC3. Values represent the percentage of the NK cells or ILCs. (B) Cells were stimulated with IL-12+IL-18 (for NK cells/ILC1), IL-25+IL-33 (for ILC2) and IL-1B+IL-23 (for ILC3), intracellular staining of IFN-y, IL-13, IL-22 were performed, and histograms are illustrated.

FIGS. 5 illustrates generation of ILCs from CD34+α4β7+ HSC progenitors in G-Rex multi-well cell culture plate. UCB-derived CD34+ HSCs were expanded for 5 days, CD34+α4β7+ progenitors were sorted by FACS and expanded for 2 more-days, CD34+α4β7+ progenitors were cultured in G-Rex multi-well cell culture plates for 3 weeks of differentiation. (A) The total number of differentiated cells are indicated for 3 or 1 million/well cells cultured in 6-well G-Rex multi-well cell culture plate. (B) The total number of differentiated cells were demonstrated for 1, 0.5, 0.2, 0.1, 0.05 or 0.025 cells/well cultured in 24-well G-Rex multi-well cell culture plate. The G-Rex system is a propriety culture flask system that gives better gas exchange than typical flasks and therefore allows cells to be cultured at higher densities, but other systems are available. The exemplary flasks provide improved gas exchange allowing use of higher cell numbers while reducing media and therefore, feeding the cells less frequently with increased efficiency. The table below illustrates how the number of cells seeded can impact the total number of ILCs. (G-Rex found at G-REX® FOR T CELL THERAPY Wilson Wolf Manufacturing)

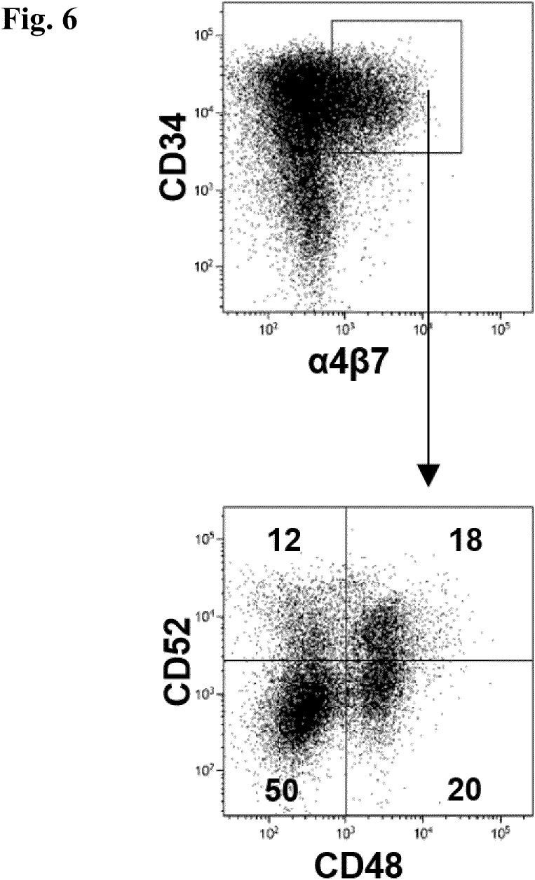

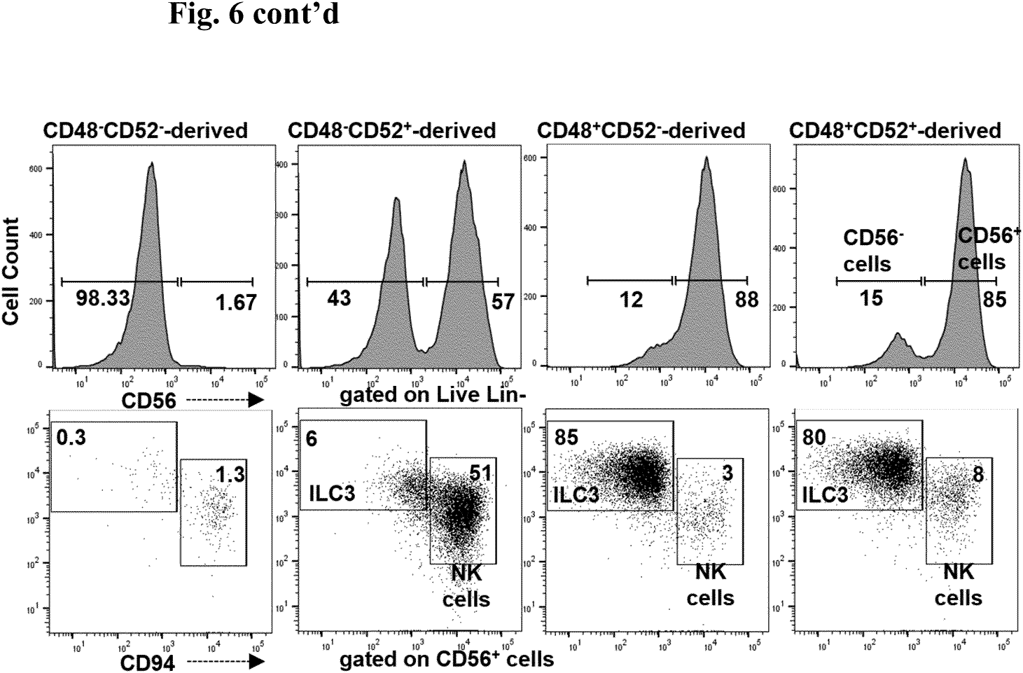

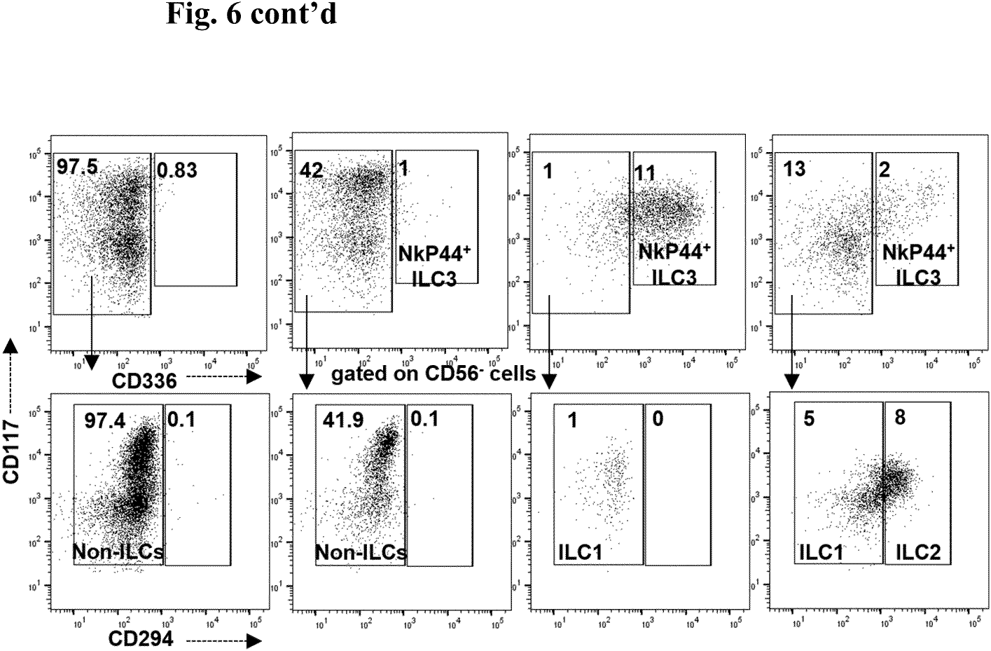

FIG. 6 illustrates data representing immature ILC precursors differentiating to give rise to mature ILCs. In this exemplary experiment, UCB-derived CD34⁺ HSCs were expanded for 5 days, and CD34⁺α4β7⁺ hematopoietic progenitors were sorted using FACS followed by twenty-one days culture under conditions that favor ILC differentiation. The output of the sorted populations (CD34⁺α4β7⁺CD48⁻CD52⁻, CD34⁺α4β7⁺CD48⁻CD52⁺, CD34⁺α4β7⁺CD48⁺CD52⁻ and CD34⁺α4β7⁺CD48⁺CD52⁺) is illustrated. Antibodies for lineage markers (containing CD1a, CD3, CD4, CD5, CD11c, CD14, CD19, CD34, TCRαβ, TCRɣδ, FcεRI, CD123, and CD303) were used to exclude any possible lineage contamination, while viability dye was used to exclude dead cells. Cells were stained for NK cell or ILCs surface markers: NK cells, ILC1, ILC2, ILC3 and NKp44⁺ ILC3 are demonstrated in dot plots, values are the percentage.

Table 1. Number of CD34⁺α4β7⁺ HSC Progenitors cultured in 24- or 6-well G-Rex multi-well cell culture plate and the number of ILCs produced in each condition.

TABLE 1

| G-Rex Plate type | Cells cultured per well day 1 (10⁶) | Wells used per donor | Plates used per donor | Cell count per well day 21 (10⁶) | Total ILCs per donor day 21 (10⁶) |
|---|---|---|---|---|---|
| 6 well | 3 | 3.5 | 1 | 12 | 34.84 |
| 6 well | 1 | 10 | 2 | 7 | 58.1 |
| 24 well | 1 | 10 | 1 | 5.7 | 47.31 |
| 24 well | 0.5 | 20 | 1 | 5.8 | 96.28 |
| 24 well | 0.2 | 50 | 3 | 2.3 | 95.46 |
| 24 well | 0.1 | 100 | 5 | 2.2 | 182.6 |
| 24 well | 0.05 | 200 | 9 | 2.1 | 348.6 |
| 24 well | 0.025 | 400 | 17 | 0.3 | 99.6 |

### Exemplary Methods

Isolation and expansion of CD34+ HSCs. In one exemplary method, mononuclear cells were isolated by density gradient centrifugation using Lymphoprep. UCB-derived CD34⁺ HSCs were positively enriched using MACS CD34⁺ enrichment kit. The cells (purity, >95%) were suspended (5×10⁴ cells/ml) in Stemspan II cell culture media supplemented with 1% penicillin + streptomycin, stem cell factor (SCF, 100 ng/ml), FMS-like tyrosine kinase 3 (Flt3L, 100 ng/ml), thrombopoietin (TPO, 50 ng/ml) and low density lipoprotein (LDL, 10 µg/ml) and cultured in 24 well plates for 5 days of expansion. After 5 days of expansion the cells were expanded three-fold on average, while the proportion of CD34⁺ cells remained >95%.

Differentiation of CD34⁺ HSCs. In another exemplary method, after 5 days of culture, the expanded cells were considered for further differentiation experiments. Where specified, expanded CD34⁺ HSCs were FACS sorted into different subsets including CD34⁺α4β7⁺, CD34⁺α4β7⁻, CD34⁺α4β7⁺CD48^{+/-} and CD34⁺α4β7⁺CD48⁺CD52^{+/-}. For up to 28 days of differentiation, cells were cultured in B0 differentiation media supplemented with SCF (20 ng/ml), IL-3 (5 ng/ml), IL-7 (20 ng/ml), IL-15 (10 ng/ml), IL-23 (10 ng/ml) and Flt3L (10 ng/ml). In some experiments where indicated cells were also cultured in the presence or absence of pre-plated and irradiated EL08.1D2 stromal cells. After a week of culture, IL-3 was excluded from the B0 differentiation media supplements. For plating progenitors on the stromal cells, 100 progenitor cells were plated per well of 96 well plates on the irradiated EL08.1D2 cells in 150 µl B0 differentiation media. Alternatively, cells were also plated without stroma using 96 well U-bottom plate and 1×10³ cells were cultured per well. Culturing, maintaining and preparation of irradiated stromal layer of EL08.1D2 cells on 96 well plate culture was as described herein.

CD244 cross-linking, blocking and knockout. To study potential stimulatory effects of CD244 activation in CD244 expressing CD48⁺ progenitors, anti-CD244 antibody clone C1.7 was used to initiate cross-linking. For this purpose, anti-CD244 or isotype IgG antibody was coated as 2 µg/ml in PBS on flat bottom 96 well culture plates for two hours at room temperature. Following blocking by 5% FBS-containing culture media and three cycles of washing with PBS, cells were plated using B0 differentiation media on the coated plates to facilitate CD244 cross-linking. After 48 hours of culture, cells were collected and transferred to a newly coated plate. Cells were finally collected after a total of 96 hours of cross-linking and plated for further differentiation in U-bottom 96 well cell culture plate. Alternatively, to activate CD244 by its natural ligand, human CD48 expressing OP9 stromal cells were generated using lentivirus transduction. The progenitor cells (100 cells) were plated per well of 96 well plates on the layer of irradiated CD48 expressing OP9 cells in 150 µl B0 differentiation media. To further investigate the stimulatory effects of CD244 signaling, both CD244 and its primary ligand CD48 were blocked in differentiation cultures using 5 µg/ml neutralizing antibodies against CD244 and CD48. Moreover, CD244 were deleted from CD34⁺α4β7⁺CD48⁺ progenitors using CRISPR-Cas9. For this purpose, 1×10⁶ cells were electroporated in Amaxa 4D-Nucleofector system with a complex of Cas9 enzyme, tracrRNA and custom CD244 or control gRNA. The Alt-R Cas9 Nuclease V3, universal tracrRNA, CD244 gRNA and non-human control gRNA were purchased from Integrated DNA Technologies. The electroporated cells were plated as 1 cell per well of 96 well plates on the layer of irradiated OP9 cells in 150 µl B0 differentiation media for further differentiation.

Flow cytometry. Flow cytometry was used to analyze α4β7⁺ ILC progenitors, CD117⁺ and CD127⁺ ILC precursors as well as mature ILCs. The gating strategy for ILCs was as shown (Data not shown, Supplementary FIGS. 1 available upon request). To evaluate the intracellular IL-13, IL-22 and IFN-γ expressions in ILCs, cells were stimulated with 10 ng/ml PMA + 1 µg/ml Ionomycin or 10 ng/ml of IL-12+IL-18, IL-25+IL-33, and IL-1β + IL-23 for overnight in the presence of 2 µg/mL Brefeldin A for the last 4 hours. For intracellular staining, cells were first stained for surface markers and fixed, followed by permeabilization and staining of intracellular proteins. For CD107a degranulation assay, sorted NK cells were incubated with K562 cells at E:T of 5:1. All flow cytometry data were acquired in LSR II and analyzed using Flowjo or Kaluza analysis software. As negative controls fluorochrome conjugated isotype-matched antibodies from the respective companies were utilized. Viability of cells was analyzed using flow cytometry with the help of fixable viability dye eFluor™ 780. All of the COMPOSITIONS and METHODS disclosed and claimed herein may be made and executed without undue experimentation in light of the present disclosure. While the COMPOSITIONS and METHODS have been described in terms of preferred embodiments, it will be apparent to those of skill in the art that variation may be applied to the COMPOSITIONS and METHODS and in the steps or in the sequence of steps of the METHODS described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A composition comprising:
isolated hematopoietic progenitor cells (HPCs) expressing CD244 and CD48; and
a composition comprising at least one of an exogenous CD48 ligand and a CD244 agonist.

2. The composition according to claim 1, wherein the at least one of an exogenous CD48 ligand or a CD244 agonist comprises a concentration of the exogenous CD48 ligand or CD244 agonist sufficient to induce CD244 signaling in the HPC cells.

3. The composition according to claim 1, further comprising at least one of a cytokine or a growth factor, wherein the at least one cytokine or at least one growth factor stimulates HPC differentiation.

4. The composition according to claim 3, wherein the at least one cytokine or growth factor comprises at least one of a stem cell factor (SCF), interleukin 3 (IL-3), interleukin 7 (IL-7), interleukin 15 (IL-15), interleukin 23 (IL-23), and FMS-like tyrosine kinase 3 ligand (FLT3L).

5. The composition according to claim 1, wherein the isolated HPCs expressing CD48 and CD244 further expresses at least one marker comprising CD34, α4β7, and CD52.

6. The composition according to claim 1, wherein the isolated HPCs expressing CD48 and CD244 comprise at least one of CD52 negative and Lin negative cells.

7. The composition according to claim 1, further comprising stroma.

8. (canceled)

9. The composition according to claim 7, wherein the stroma comprises at least one of stroma that expresses or overexpresses CD48 and irradiated stroma.

10. (canceled)

11. The composition according to claim 1, wherein the at least one CD244 agonist or CD48 ligand comprises at least one of a small molecule, a peptide, a polynucleotide, a genetically modified cell expressing a CD244 agonist or CD48 ligand, an antibody, an antibody fragment capable of at least one of activating the CD244, inhibiting CD48/2B4 interaction, or a combination thereof.

12. The composition according to claim 1, wherein the hematopoietic progenitor cell expresses CD244 receptors and the at least one CD48 ligand, CD48 agonist or CD48 antagonist modulates activation of the CD244 receptors.

13. A method for increasing in vitro production of ILC2 cells comprising providing to an isolated hematopoietic progenitor cell (HPC) population expressing CD48 and CD244, a composition comprising at least one of a CD48 ligand or a CD244 agonist and producing increased ILC2 cells compared to a control HPC population not exposed to the CD48 ligand, the CD244 agonist or the CD48 antagonist.

14. The method according to claim 13, further comprising seeding the HPC expressing CD48 and CD244 onto a population of stromal cells.

15. (canceled)

16. The method according to claim 14, wherein the stromal cells comprises stromal cells including at least one of stromal cells that express CD48 and irradiated stromal cells.

17. (canceled)

18. The method according to claim 13, further comprising providing to the hematopoietic progenitor cell expressing CD48 and CD244, at least one of cytokines, or a growth factor, wherein the at least one cytokine or growth factor stimulates hematopoietic progenitor cell differentiation.

19. The method according to claim 18, wherein the at least one cytokines or growth comprises stem cell factor (SCF), interleukin 2 (IL-2), interleukin 3 (IL-3), interleukin 7 (IL-7), interleukin 15 (IL-15), interleukin 23 (IL-23), interleukin 25 (IL-25), interleukin 33 (IL-33), and FMS-like tyrosine kinase 3 ligand (FLT3L).

20. The method according to claim 13, wherein the ILC2 cells comprises at least one of ILC2 cells expressing CD11a, CD117, or a combination thereof and enriched ILC2 cells, wherein the induced HPCs comprise about 50% or more ILC2 cells.

21. (canceled)

22. A kit comprising the composition according to claim 1, and at least one container.

23. A method for treating a health condition in a subject, the method comprising administering a composition comprising ILC2s to the subject, wherein the ILC2s comprise ILC2s produced by the method according to claim 13.

24. The method of claim 23, wherein the health condition comprises at least one of: graft versus host disease (GvHD), a cardiac condition, a renal condition, an inflammatory bowel diseases (IBD), Type-1 Diabetes, psoriasis, asthma, allergies, an immune-related condition, rheumatoid arthritis, ankylosing spondylitis, psoriasis, psoriatic arthritis, Behcet’s disease, viral infections, or a combination thereof.

25. The method of claim 24, wherein one or more immune-related conditions comprises cancer.

26-27. (canceled)
